SEQUENCE LISTING (10 sequences)

SEQ ID NO: 1 - DNA, 19 bp, Artificial Sequence
  Primer
atcgtgatgtagatagtcg

SEQ ID NO: 2 - DNA, 20 bp, Artificial Sequence
  Primer
ctagctagctggtaccatga

SEQ ID NO: 3 - DNA, 2151 bp, MOUSE IDPc
gagctaactggggccggcttattacagcttgtgtgtacgcgcgggtgtgagccgggttattgaagtaaaaatgtccagaaaaatccaaggaggttctgtggtggagatgcaaggagatgaaatgacacgaatcatttgggaattgattaaggaaaaacttattcttccctatgtggaactggatctgcatagctatgatttaggcatagagaatcgtgatgccaccaatgaccaggtcaccaaagatgctgcagaggctataaagaaatacaacgtgggcgtcaagtgtgctaccatcacccccgatgagaagagggttgaagaattcaagttgaaacaaatgtggaaatccccaaatggcaccatccgaaacattctgggtggcactgtcttcagggaagctattatctgcaaaaatatcccccggctagtgacaggctgggtaaaacccatcatcattggccgacatgcatatggggaccaatacagagcaactgattttgttgttcctgggcctggaaaagtagagataacctacacaccaaaagatggaactcagaaggtgacatacatggtacatgactttgaagaaggtggtggtgttgccatgggcatgtacaaccaggataagtcaattgaagactttgcacacagttccttccaaatggctctgtccaagggctggcctttgtatctcagcaccaagaacactattctgaagaagtatgatgggggtttcaaagacatcttccaggagatctatgacaagaaatacaagtcccagtttgaagctcagaagatctgctatgaacacaggctcatagatgacatggtggcccaagctatgaagtccgagggaggcttcatctgggcctgtaagaattacgatggggatgtgcagtcagactcagtcgcccaaggttatggctcccttggcatgatgaccagtgtgctgatttgtccagatggtaagacggtagaagcagaggctgcccatggcactgtcacacgtcactaccgcatgtaccagaaagggcaagagacgtccaccaaccccattgcttccatttttgcctggtcccgagggttagcccacagagcaaagcttgataacaatactgagctcagcttcttcgcaaaggctttggaagacgtctgcattgagaccattgaggctggctttatgactaaggacttggctgcttgcattaaaggcttacccaatgtacaacgttctgactacttgaatacatttgagtttatggacaaacttggagaaaacttgaaggccaaattagctcaggccaaactttaaggtcaaacctgggcttagaatgagtctttgcggtaactaggtccacaggtttacgtattttttttttttttttagtaacactcaagattaaaaaaaaaaatcattttgtaatttgtttagaagacaaagttgaacttttatatatgtttacagtcttttttctttttcatacagttattgccaccttaatgaatgtggtggggaaatttttttaattgtattttattgtgtagtagcagtgtaggaattatgttagtacctgttcacaattaactgtcatgttttctcatgctctaatgtaaatgaccaaaatcagaagtgctccaagggtgaacaatagctacagtatggttccccataaggggaaaagagaaactcacttcccctgttgtccatgagtgtgaacactggggcctttgtacgcaaatgttgtactgtgtgtgggagagctatacagtaagctcacataagactggaacagataggatgtgtgtagctaaaatgcatggcagacgtgtttataaagagcatgtatgtgtccaatatactagttatattttaagaccactggagaattccaagtctagaataaatgcagactggaggattctgctctttgatttctcttctcctgtgacccagcctaagtattatcctaccccaagcagtacatttcacccatgggcaataatgggagctgtaccgtttggatttctgctgacctgctgcatttcttttatataaatgtgacttttttttcccagaagttgatattaaacactattccagtctagtccttctaaactgttaattttaattaaaatgaagtactaatg

SEQ ID NO: 4 - protein, 414 aa, MOUSE IDPc
MSRKIQGGSVVEMQGDEMTRIIWELIKEKLILPYVELDLHSYDLGIENRDATNDQVTKDAAEAIKKYNVGVKCATITPDEKRVEEFKLKQMWKSPNGTIRNILGGTVFREAIICKNIPRLVTGWVKPIIIGRHAYGDQYRATDFVVPGPGKVEITYTPKDGTQKVTYMVHDFEEGGGVAMGMYNQDKSIEDFAHSSFQMALSKGWPLYLSTKNTILKKYDGGFKDIFQEIYDKKYKSQFEAQKICYEHRLIDDMVAQAMKSEGGFIWACKNYDGDVQSDSVAQGYGSLGMMTSVLICPDGKTVEAEAAHGTVTRHYRMYQKGQETSTNPIASIFAWSRGLAHRAKLDNNTELSFFAKALEDVCIETIEAGFMTKDLAACIKGLPNVQRSDYLNTFEFMDKLGENLKAKLAQAKL

SEQ ID NO: 5 - DNA, 22 bp, Artificial Sequence
  Primer
agatctccttgactaatataac

SEQ ID NO: 6 - DNA, 20 bp, Artificial Sequence
  Primer
taatacgactcactataggg

SEQ ID NO: 7 - DNA, 20 bp, Artificial Sequence
  Primer
ctagctaccaagcacggttg

SEQ ID NO: 8 - DNA, 20 bp, Artificial Sequence
  Primer
tcagttgctctgtattggtc

SEQ ID NO: 9 - DNA, 19 bp, Artificial Sequence
  Primer
ggccaacaggggaaatccg

SEQ ID NO: 10 - DNA, 24 bp, Artificial Sequence
  Primer
gctctagaaatccttgactaatat

(19) United States
(12) Patent Application Publication
(10) Pub. No.: US 2004/0214270 A1
(43) Pub. Date: Oct. 28, 2004

(54) Isocitrate dehydrogenase, gene thereof, and use of the same in the treatment of obesity, hyperlipidemia, and fattly liver in lipid biosynthesis

(76) Inventors: Tae-Lin Huh, Daegu (KR); Ho-Jin Koh, Daegu (KR); Myung-Sook Choi, Daegu (KR); Un-Ju Jung, Daegu (KR)

Correspondence Address: JHK LAW, P.O. BOX 1078, LA CANADA, CA, 91012-1078, US

(21) Appl. No.: 10/239,800

(22) PCT Filed: Jul. 26, 2001
(86) PCT No.: PCT/KR01/01271

(30) Foreign Application Priority Data
Oct. 20, 2000 (KR) 2000/61962

(51) Int. Cl. C12N 9/02; C12P 21/02; C12N 5/06; C07H 21/04

(52) U.S. Cl. 435/69.1; 435/189; 435/320.1; 435/325; 536/23.2

(57) ABSTRACT

The present invention relates to a cytosolic isocitrate dehydrogenase, its gene, and its use in the treatment of obesity, hyperlipidemia, and fatty liver. The expression of the IDPc gene and the concomitant increase in IDPc level bring about an increase in the cellular level of NADPH, which causes the lipid deposition in adipocytes, leading to obesity and fatty liver. A decrease in the cellular level of NADPH, resulting from the suppression of the gene expression of IDPc, has the effect of inhibiting the lipid deposition in adipocytes. Further, by taking advantage of the suppressive or inhibitory effects of isocitrate dehydrogenase inhibitors, pharmaceutically effective materials for the prophylaxis and treatment of obesity, hyperlipidemia and fatty liver can be developed.

### CONTINUING DATA

The present application is a national stage application of PCT/KR01/01271, filed Jul. 26, 2001 under 35 U.S.C. 371.

### FIELD OF THE INVENTION

The present invention relates to an isocitrate dehydrogenase which catalyze the production of NADPH necessary for the biosynthesis of lipids, including fatty acids, squalene and cholesterol, and its use in the treatment of metabolic diseases, including obesity, hyperlipidemia and fatty liver. Also, the present invention relates to an isocitrate dehydrogenase gene, fused gene constructs containing the gene, transfectant cells harboring the genes in their genome, and transgenic animals capable of expressing isocitrate dehydrogenase continuously throughout their lifespan.

### BACKGROUND OF THE INVENTION

Taking part in the TCA (tricarboxylic acid) cycle, isocitrate dehydrogenase catalyses the oxidative decarboxylation of citric acid into α-ketoglutarate with concurrent production of NADH or NADPH.

In higher animals, isocitrate dehydrogenase isozymes can be separated into three classes according to their cofactors and locations in the cell: mitochondrial NAD⁺-dependent isocitrate dehydrogenase (hereinafter referred to as “IDH”), mitochondrial NADP⁺-dependent isocitrate dehydrogenase (hereinafter referred to as “IDPm”), and cytoplasmic NADP⁺-dependent isocitrate dehydrogenase (hereinafter referred to as “IDPc”). Among these isocitrate isoenzymes, IDH has been assumed to play a major role in the oxidative decarboxylation of isocitrate in the tricarboxylic acid cycle (TCA) with concurrent production of α-ketoglutarate and NADH. NADH is used for energy generation through the electron transfer system and α-ketoglutarate is a metabolite used in the synthesis of amino acids such as glutamic acid, glutamine, arginine, and proline, and other biological products. IDH activity is regulated as a control point of the TCA cycle. Therefore, IDH is a key enzyme to regulate not only the TCA cycle, but also energy metabolism, protein biosynthesis and nitrogen metabolism because metabolites of the TCA cycle take part in such metabolisms.

Since its isolation from yeast and pig, IDH has been under study. Yeast IDH is an allosterically regulated enzyme that exists as an octamer composed of two nonidentical subunits IDH1 and IDH2 sharing high homology with each other. IDH1 plays a role in the regulation of the enzyme activity while IDH2 is responsible for the catalytic activity (Keys, D. A. & McAlister-Henn, L., J. Bacteriol., 172, 4280-4287, 1990). Broken down into three subunits (α, β, γ subunits), swine IDH also exists as an octamer (2(α2β γ)) in active form.

Found to have bipartite structures, IDPm and IDPc are, however, not known as to their functions. Although both having molecular weight of about 45 kDa with high homology, the two enzymes were identified as different, independent proteins, as analyzed by immunological reaction experiments using polyclonal antibodies (Plaut, G. W. E. et al., Biochem. Biophys. Acta., 760, 300-308, 1983; Fantania, H. R. et al., FEBS, 322, 245-248, 1993). Particularly, IDPm and IDPc are highly tissue-specific. In cardiac muscle tissues, for instance, more than 90% of total NADP⁺-dependent isocitrate dehydrogenase exists in mitochondria and the remaining 10% is found in cytoplasm. In contrast, it is reported that as low as 3% of the total NADP⁺-dependent isocitrate dehydrogenase of liver tissues is found in mitochondria while the remaining 97% exists in cytoplasm (Plaut, G. W. E., Current Topics in Cell Regulation, 2, 1-27, 1983).

As mentioned above, isocitrate dehydrogenase isozymes have been characterized concerning some of their structural characteristics, but not concerning functions. Particularly, nowhere had been found studies on precise mechanisms of IDPm and IDPc until the publication of recent reports which merely made the assumption that IDPm catalyzes a reverse reaction in the TCA cycle to convert α-ketoglutarate through isocitrate to citrate, which is associated with a tricarboxylate carrier to supply acetyl-CoA, a precursor for the biosynthesis of fatty acids and cholesterol, with concurrent conversion of the citrate to oxaloacetate to raise cytoplasmic phosphoenolpyruvate levels, thereby promoting gluconeogenesis (Des Rosiers, C. et al., J. Biol. Chem., 269, 27179-27182, 1994; Fernandez, C. A. et al., J. Biol. Chem., 270, 10037-10042, 1995).

Significance in gluconeogenesis is suggested for IDPm owing to its catalysis of a reverse reaction of the TCA cycle. In contrast, none of the reports for IDPc are concerned with its metabolic functions. IDPc is known to be expressed in large quantities in the ovary and the mammary gland. Of the NADPH producing enzymes existing in rat liver, IDPc has been quantitatively analyzed to produce NADPH in greater quantities than do important enzymes of the pentose phosphate pathway; i.e., glucose-6-phosphate dehydrogenase for the conversion of glucose-6-phosphate to 6-phosphoglucono-δ-lactone and NADPH, 6-phosphogluconate dehydrogenase for the conversion of 6-phosphogluconate to ribulose-5-phosphate and NADPH, and cytoplasmic malic enzyme for the conversion of malate to pyruvate and NADPH; by factors of 16, 8 and 18, respectively (Veech, R. L. et al., Biochem. J., 115, 609-619, 1969).

In cytoplasm, various enzymes involved in metabolisms of fatty acids, cholesterol and hormones require a large quantity of NADPH for their catalytic activities. Thus far, the NADP producing enzymes such as glucose-6-phosphate dehydrogenase and malic enzyme have been believed to play an important role in supplying NADPH to cytoplasm. However, in light of its ability to produce cytoplasmic NADPH, IDPc is expected to be more responsible for the regulation of the supply of NADPH. Ultimately, it is assumed that IDPc plays a crucial role in the biosynthesis of fatty acids and cholesterol. Among the fatty acid synthases implicated in the biosynthesis of fatty acids, β-ketoacyl-ACP reductase and enoyl-ACP reductase require NADPH as a cofactor for their catalysis. In the biosynthesis of cholesterol, a large quantity of NADPH is required for the reactions catalyzed by HMG-CoA reductase and squalene synthetase and for the final 19-step reaction from lanosterol to cholesterol. Accordingly, control of the activity of IDPc, which functions to supply most of the NADPH required in the cell, is very important to regulate the biosynthesis of fatty acids and their derivatives, lipids, squalene, and cholesterol and its derivatives.

In higher animals, lipid deposition follows the following procedure. When excess energy sources are available, the differentiation of adipose cells is accelerated, resulting in an increase in the number and size of white adipose tissues with concomitant deposition of lipids. In turn, the white adipose tissue allows the ob gene to be actively expressed, which leads to an increase in body leptin level. In response, the hormonal action in the brain is changed toward the decreasing of appetite. Meanwhile, excess calories are consumed to maintain the body temperature, using uncoupler proteins (UCP). In the white adipose tissue, expression of the genes which encode master transcription factors for the proliferation of adipose cells, such as peroxysome proliferator-activated receptor γ (PPARγ), C/EBPα and ADD1/SREBP1, is activated. Thus, adipose cell differentiation and lipid deposition are promoted and excess body energy is stored in lipid form, so that body energy is balanced (Hu, E. et al., Proc. Natl. Acad. Sci. USA, 92, 9856-9860, 1995; Keller, H. et al., Proc. Natl. Acad. Sci. USA, 20, 9856-9860, 1993; Freytag S. O., et al., Genes Dev., 8, 1654-1663, 1994; Tontonoz, P. et al., Mol. Cell. Biol., 13, 4753-4759, 1993; Spiegelman, B. M., Cell, 87, 377-389, 1996). Examples of ligands necessary for the activation of PPARγ, a master transcription factor for adipose cell differentiation, include polyunsaturated fatty acids such as linoleic acid, docosahexanoic acid (DHA), and arachidonic acid (Krey, G. et al., Mol. Endocrinol., 11, 779-791, 1997; Yu et al., J. Biol. Chem., 270, 23975-23983, 1995). Also, prostaglandin J2 is known to serve as a ligand of the master transcription factor (Forman B. M. et al., Cell, 83, 803-812, 1995; Kliewer S. A. et al., Cell, 83, 813-819, 1995).

From this review, there is concluded a high possibility that IDPc might be directly involved in controlling the biosynthesis of various fatty acids, cholesterol and hormones owing to its ability to produce NADPH. Also, IDPc can be assumed to play a key role in obesity and fatty liver by encouraging the production of activating ligands for PPARγ, such as polyunsaturated fatty acids and arachidonic acid to trigger the cascade expression of various genes related to the differentiation of adipose cells. Additionally, the requirement of a large quantity of NADPH for cholesterol biosynthesis offers the possibility that artificial control of intracellular levels of IDPc and its reaction product NADPH might provide a means of controlling cholesterol biosynthesis.

### SUMMARY OF THE INVENTION

Leading to the present invention, the intensive and thorough research on the mechanism of lipid biosynthesis through molecular biological and biochemical experiments using transfectant animal cells and transgenic mice, conducted by the present inventors, resulted in the finding that intracellular levels of IDPc and its reaction product NADPH have a decisive influence on not only the differentiation rate of adipose cells and lipid deposition in adipose cells, but the biosynthesis of lipids and cholesterol.

Therefore, it is an object of the present invention to provide an isocitrate dehydrogenase enzyme for producing NADPH, and its gene.

It is another object of the present invention to provide a fused gene construct which contains a gene encoding isocitrate dehydrogenase, a transfectant cell which harbors the gene in its genome, and a transgenic animal which can express the gene continuously throughout its lifespan.

It is a further object of the present invention to provide the use of isocitrate dehydrogenase and its gene in the treatment and prophylaxis of obesity, hyperlipidemia, and fatty liver or in the biosynthesis of lipids.

### BRIEF DESCRIPTION OF THE DRAWINGS

FIG. 1 provides schematic diagrams showing structures of a basic LNCX-vector (top), a recombinant vector into which an IDPc gene is inserted in the sense orientation to increase the expression of the IDPc gene in NIH3T3 F442A adipocytes (middle), and a recombinant vector into which an IDPc gene is inserted in the antisense orientation to decrease the expression of the IDPc gene in NIH3T3 F442A adipocytes (bottom).

FIG. 2a provides optical photographs showing Oil-Red-O-dyed adipocytes differentiated from normal NIH3T3 F442A (left), the transfectant FS1 cells with improved IDPc gene expression (middle), and the transfectant FAS1 cells with decreased IDPc gene expression (right) on plates (upper panel) and in part, magnified at 200 power (lower panel).

FIG. 2b provides optical photographs showing the lipid deposition in adipocytes, which is in a NADPH dose-dependent pattern.

FIG. 3 is a diagram illustrating the construction of a recombinant expression vector for use in generating a transgenic animal, in which an IDPc cDNA is inserted downstream of a rat-derived PEPCK (phosphoenolpyruvate carboxykinase) gene promoter.

FIG. 4 provides photographs showing a comparison in body size and epididymal fat pad deposit between F₁ progeny from the transgenic mice of the present invention and normal mice.

FIG. 5 provides autoradiographs showing an increase in the expression level of obesity-indicative genes in the adipose tissue of the transgenic mice of the present invention, compared to normal mice.

FIG. 6a is a histogram comparing the body weight of the transgenic mice F₁ to that of normal mice.

FIG. 6b is a histogram comparing the liver weight of the transgenic mice F₁ to that of normal mice.

FIG. 6c is a histogram comparing the IDPc activity and blood IDPc level of the transgenic mice F₁ to those of normal mice.

FIG. 6d is a histogram comparing the [NADPH]/[NADPH+NADP⁺] of the transgenic mice F₁ to that of normal mice.

FIG. 6e is a histogram comparing the epididymal fat pad weight of the transgenic mice F₁ to that of normal mice.

FIG. 6f is a histogram comparing the blood triglyceride and cholesterol levels of the transgenic mice F₁ to those of normal mice.

FIG. 6g is a histogram comparing the triglyceride and cholesterol levels in the liver of the transgenic mice F₁ to those of normal mice.

FIG. 6h is a histogram comparing the blood leptin level of the transgenic mice F₁ to that of normal mice.

FIG. 7a provides photographs showing liver tissues of the transgenic mice of the present invention and the control mice.

FIG. 7b provides photographs showing adipocyte of the transgenic mice of the present invention and the control mice.

FIG. 8a is a graph illustrating the inhibitory activity of oxalomalic acid against isocitrate dehydrogenase activity.

FIG. 8b is a graph illustrating the inhibitory activity of methyl isocitrate against isocitrate dehydrogenase activity.

FIG. 9 provides optical photographs showing Oil-Red-O-dyed adipocytes differentiated from NIH3T3 F442A cell treated with no isocitrate dehydrogenase inhibitors (left), oxalomalate (middle), and methyl isocitrate (right), magnified at 100 power (upper panel) and 200 power (lower panel).

FIG. 10a is a histogram illustrating comparing the weights of the liver and epididymal fat pad of the rats in which the isocitrate dehydrogenase inhibitor of the present invention is administered, to those of rats administered with no inhibitors.

FIG. 10b is a histogram illustrating comparing the blood triglyceride and cholesterol levels of the rats into which the isocitrate dehydrogenase inhibitor of the present invention is administered, to those of non-administered rats.

FIG. 10c is a histogram illustrating comparing the blood HDL level of the rats into which the isocitrate dehydrogenase inhibitor of the present invention is administered, to that of non-administered rats.

### DETAILED DESCRIPTION OF THE INVENTION

In an aspect, the present invention pertains to an isocitrate dehydrogenase enzyme which catalyzes the production of NADPH necessary for the biosynthesis of fatty acids and cholesterol and the deposition of lipids, and to a gene encoding the isocitrate dehydrogenase.

Useful in the present invention is the IDPc isolated from mice. The mouse-derived IDPc gene of the present invention, as listed in Sequence No. 3, has an open reading frame (ORF) 1,245 bp in size, with a 3′-untranslated region (UTR) in which a base sequence AATAAA, a putative poly-A signal, exists. The IDPc protein for which the IDPc gene codes consists of 414 amino acids, listed in Sequence No. 4, with a molecular weight of 46,575 Da. Alignment of the IDPc amino acid sequences from various species indicates that the mouse IDPc of the present invention shares a homology of 97.8% with rat IDPc, 68.5% with bovine IDPm, and 64.4% with yeast IDPc. Particularly, an amino acid sequence from 412 to 414 of the mouse IDPc is identical to the target sequence of peroxisome, which is known to be involved in the biosynthesis and degradation of fatty acids and cholesterol. Therefore, this suggests the high possibility that IDPc moves to peroxisomes and takes part in the synthesis of fatty acids and cholesterol thereat.

In addition to IDPc, IDPm, a gene having a base sequence similar to that of the IDPc gene, is also used for producing NADPH required for the biosynthesis of fatty acids and cholesterol and the deposition of lipids in accordance with the present invention.

In another aspect, the present invention pertains to a fused gene construct containing the gene, a novel cell strain which anchors the gene, and a transgenic animal which expresses the gene continuously throughout its lifespan.

To this end, first, the gene of interest is inserted into a mammalian expression vector in such a way as to transcribe the gene in the sense direction or in the antisense direction.

In this regard, retroviral expression vectors are preferably used as the gene carrier, with highest preference for pLNCX retroviral vector. pLNCX, which is derived from MMLV (Moloney murine leukemia virus), has a CMV (cytomegalovirus) promoter for expressing exogenous genes in mammalian cells, and a neomycin gene as a selection marker, along with an LTR (long terminal repeat) sequence, an identification factor of retroviral vectors.

Among the fused gene constructs thus prepared, those which are transcribed in the sense direction by the CMV promoter are used to enhance the expression of IDPc, while those designed for antisense transcription are used to suppress the expression. Resultant recombinant vectors are introduced into NIH3T3 L1 cells, a kind of preadipocytes. Of the cell transfectants which were identified to have integrated the IDPc gene into their genomes, the cells in which IDPc gene were inserted in the sense direction were named FS1, which was deposited with the Korean Collection for Type Culture of Korea Research Institute of Bioscience and Biotechnology (KRIBB) under the deposition No. KCTC 0861BP, on Sep. 6, 2000. On the other hand, cell strains which have the IDPc gene inserted in the antisense direction were named FAS1.

Compared to the mouse NIH3T3 L1 cells into which only the pLNCX vector was introduced (control), the enzyme activity was measured to be higher by about 2 fold in the mouse NIH3T3 L1 transfectant cell in which the IDPc gene was inserted in the sense direction (FS1), but lower by about 0.4 fold in the mouse NIH3T3 L1 transfectant cells in which the IDPc gene was inserted in the antisense direction (FAS1).

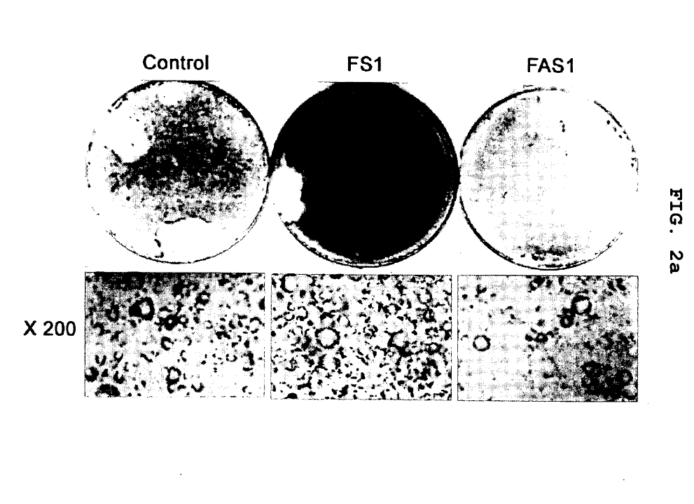

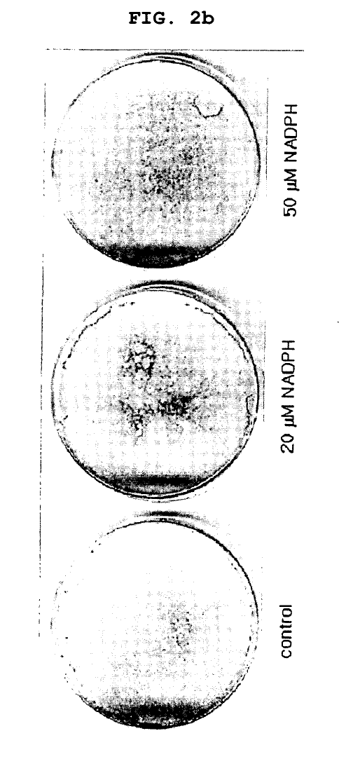

The effect of IDPc on the biosynthesis of fatty acids can be quantitatively measured with Oil-Red-O, a dye specific for lipids, which is applied to the adipocytes which have been differentiated from the transfectant cells after treatment with insulin. As a result, lipid production was found to be conducted more actively in the transfectant cell FS1 with improved IDPc gene expression than the control cell. On the other hand, little deposition of lipids was found in the transfectant cells FAS1 with lowered IDPc gene expression, compared to the control cell (see FIG. 2a). While being differentiated to adipocytes in the presence of NADPH, which is an enzymatic reaction product of IDPc, the transfectant cells into which only pLNCX was introduced, that is, control cells, show higher differentiation rates and larger intracellular lipid deposits as the concentration of NADPH increases (see FIG. 2b). These results indicate that IDPc, its gene, or its enzymatic reaction product NADPH plays a key role in determining intracellular lipid deposits.

Next, in order to examine the activity of isocitrate dehydrogenase, the fused gene construct is used to prepare a transgenic animal which harbors the IDPc gene within its genome.

1. Preparation of Fused Gene Construct

To express the IDPc gene permanently, there is required the integration of the gene into the genome of an animal. To this end, first, it is necessary to construct a recombinant vector which can express the gene of interest in mammals. In the resulting fused gene construct, the expression of the gene of interest is regulated under a suitable promoter gene. The term “fused gene construct” as used herein, means a functional assembly of genes for use in transformation of certain organisms, which is comprised essentially of at least one structural gene, and at least one cis-acting regulatory element for controlling the expression of the structural gene.

Generally, a cis-acting regulatory element may be in the form of a promoter, an enhancer, an intron, a 5′-UTR (untranslated region), and a 3′-UTR. In a fused gene construct, the cis-acting regulatory element may be located at any site of 10 kb or less distant from the 5′-flanking region, 3′-flanking region, 5′-end or 3′-end of the structural gene or inside the structural gene (in the case of an intron). In addition to the structural gene and cis-acting regulatory element, the fused gene construct further comprises various components, including a polyadenylation signal for improving transcription or translation rates, a ribosome-binding sequence, an intron, etc. Further to these, a base sequence for improving the efficiency of the insertion of a gene of interest into the genome or certain sites, and a marker gene for identifying the insertion may be provided for the fused gene construct.

A promoter for the fused gene construct to be used in making a transgenic animal include the CMV promoter, or expression regulatory regions for genes expressible in white adipose tissues, such as genes coding for lipoprotein lipase (LPL), adipsin, adipocyte protein 2 (aP2) and IDPc. In a preferred embodiment of the present invention, there is employed a rat-derived promoter for a cytosolic phosphoenolpyruvate carboxykinase (PEPCK) gene, which is expressed in both the liver and the white adipose tissues.

In more detail, the preparation of a transgenic animal in which the permanent expression of the IDPc gene is conducted starts with the cytosolic PEPCK gene of rats. From this gene, a 2.2 kb 5′-upstream sequence containing a promoter was obtained. Downstream of this sequence, a mouse IDPc cDNA was inserted in the sense orientation to prepare a fused gene construct, which was named pPEPCKIDPc. There are two kinds of PEPCK genes: one codes for a cytosolic enzyme and the other for a mitochondrial enzyme. In the present invention, a 5′-upstream sequence of the gene encoding the cytosolic PEPCK (hereinafter referred to as “PEPCK-C”) was employed. In the liver, the intestine and the kidney, the tissues, where the PEPCK-C gene is expressed under the regulation of the promoter, are determined depending on the regulatory regions existing in the 5′-upstream sequence. The 2.2 kb 5′-upstream sequence of the PEPCK-C gene used in the present invention contains a gene sequence near nt 987, which is known as a regulatory region necessary for efficient expression in white adipose tissue (Hanson, R. W. Annu. Tev. Biochem., 66, 581-611, 1997).

Mice are useful for making transgenic animals, but any animal, if it can be made transgenic, is available in the present invention because IDPc is an enzyme expressed in all higher animals.

2. Preparation of Embryo

One of the most important steps in making of a transgenic animal is to introduce the fused gene construct into an embryo. The introduction is conducted with the aid of a microinjection system. When microinjecting the fused gene construct to an embryo, an automatic microinjection system which is able to automatically control amounts of DNA to the limit of 4 pl is preferably used because of it being superior in success rate to conventional manual microinjection systems. The mouse embryo which contains the IDPc fused gene construct was deposited with the Korean Collection for Type Culture of Korea Research Institute of Bioscience and Biotechnology (KRIBB) under the deposition No. KCTC 0874 BP, on Nov. 4, 2000.

3. Preparation of Transgenic Animal

Next, the embryo containing the fused gene construct is implanted into a surrogate mother to afford a transgenic animal. In the present invention the implantation of the embryo into a surrogate mother is conducted at the one-cell stage of the embryo rather than the two-cell stage, for convenience. Immediately after the microinjection of the fused gene construct, the embryo of the one-cell stage is implanted to the oviduct of a surrogate mother, so as to reduce various processes necessary to culture the embryo to the two-cell stage. For implantation into an oviduct at the two-cell stage, for instance, an embryo is required to be cultured for one additional day in an incubator. In order to implant a two-cell stage embryo to the oviduct funnel, the embryo must be inserted deep into the oviduct, or it is necessary to perforate the oviduct by use of a needle. However, the implantation of the one-cell stage embryo to a surrogate mother may be conducted under conditions similar to those for general mouse embryos, although the implantation site is the oviduct funnel.

Using the transgenic animal thus made, the in vivo activity of IDPc was examined in terms of the following indicators:

1. Enlargement of Epididymal Fat Pad

23 weeks after birth, F₁ heterozygous transgenic mice had grown bigger than control mice. When being anatomized, F₁ heterozygous transgenic mice were measured to be significantly increased in the size of the epididymal fat pad with a body weight 14 times as heavy as that of the control mice. Additionally, when being frayed, the transgenic mice were observed to have large and many dermal mast cells on their backs, compared to control mice. Upon complete removal of the abdominal skin, a significant increase in epididymal fat pad was observed in the transgenic mice (see FIG. 4).

2. Expression Rate of Obesity-Indicative Gene

An examination is made as to whether the expression of IDPc gene has influence on the expression of obesity-indicative genes. In the epididymal fat pad of the IDPc gene-transgenic mice, the expression of the recombinant IDPc gene introduced was found, along with the expression of their endogenous IDPc gene, demonstrating that the total IDPc expression was increased. Additionally, an increase was found in the expression of obesity-indicative genes, such as genes coding for adipocyte protein 2 (aP2), adipsin, lipoprotein lipase (LPL), leptin, tumor necrosis factor α (TNF-α), and peroxysome proliferator-activated receptor γ (PPARγ), which are all known to show increased expression with advance in the differentiation of mast cells (Hwang, C. S. et al., Ann. Rev. Cell Eev. Biol., 13, 231-259, 1997; Lemberger, T. et al., Annu. Rev. Cell Dev. Biol., 12, 225-362, 1996; Spiegelman, B. M. et al., Cell, 87, 377-389, 1996).

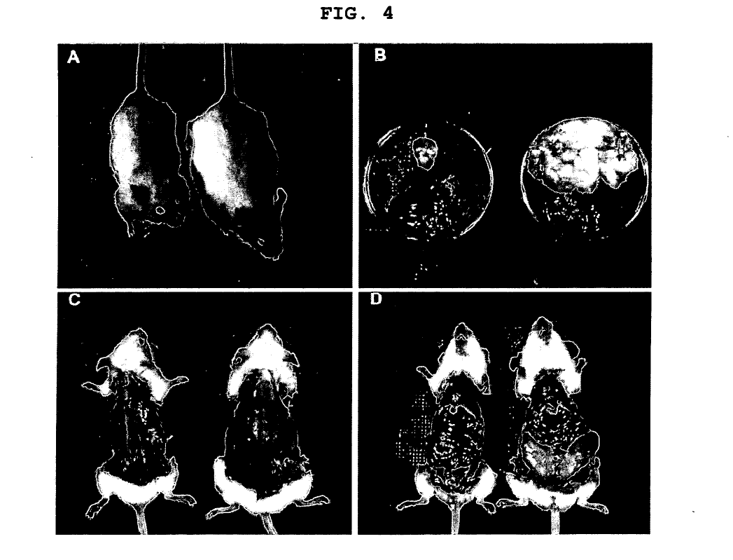

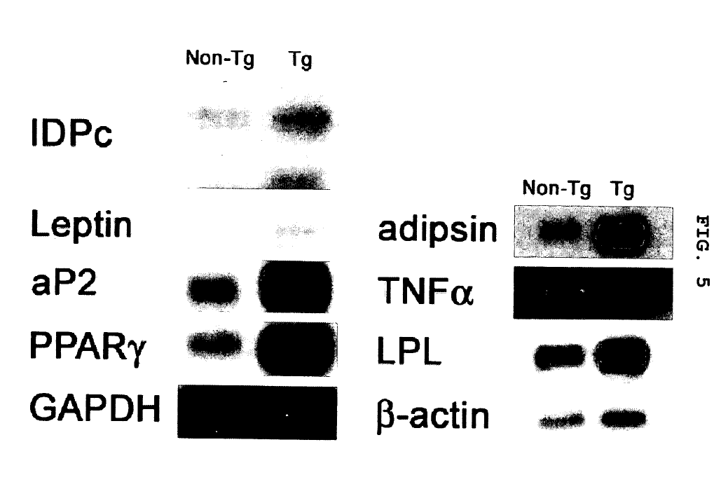

In light of the recent report revealing that PPARγ serves as a master transcription factor in both the differentiation of mast cells and the biosynthesis of lipids (Spiegelman, B. M., et al., Cell, 87, 377-389, 1996), an increase in the expression of obesity-indicative genes, such as genes encoding ap2, adipsin, LPL, leptin, and/or TNFα, in the IDPc-transgenic mice results from an increase in the expression of the PPARγ gene. Therefore, it can be concluded that an increase in IDPc activity attributed to the active expression of the IDPc gene and a concomitant increase in NADPH level causes a sharp increase in the gene expression of PPARγ, which is indispensable for both the differentiation of adipocytes and the biosynthesis of lipids (see FIG. 5). In turn, these results, when account is taken of the report disclosing that an increase in the level of the ligand necessary for the activation of PPARγ stimulates the expression of the PPARγ gene itself (Kim, J. B. et al., Proc. Natl. Acad. Sci. USA., 95, 4333-4337, 1998), leads to a further conclusion that an increase in the activity of IDPc and in the level of NADPH, which is a product of the enzyme, primarily stimulates the production of polyunsaturated fatty acids that serve as ligands capable of inducing the activation of PPARγ and secondarily induces the expression of the PPARγ gene itself in return, thereby raising the expression level of differentiation-indicative genes, such as genes coding for ap2, adipsin, LPL and leptin, which are involved in the differentiation of various adipocytes, and finally causing obesity and fatty liver.

3. Identification of Lipid Deposit in the Body

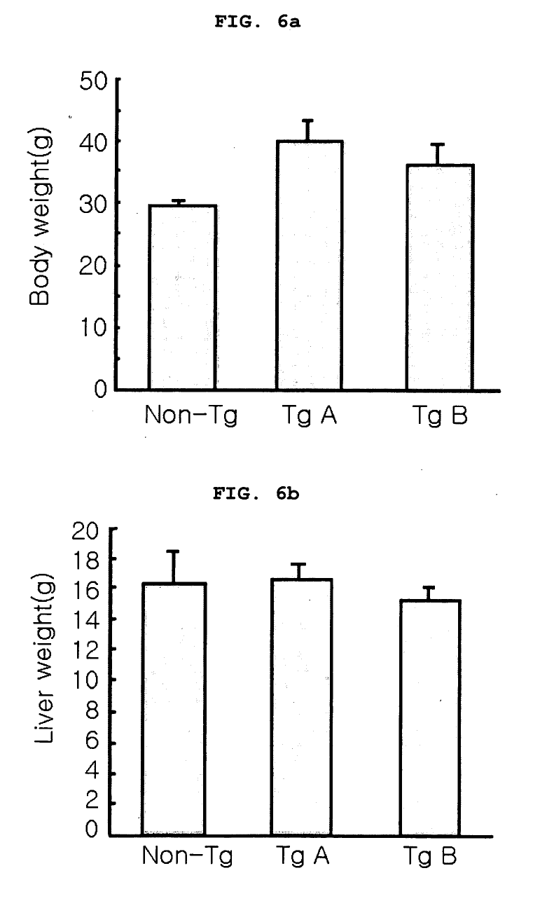

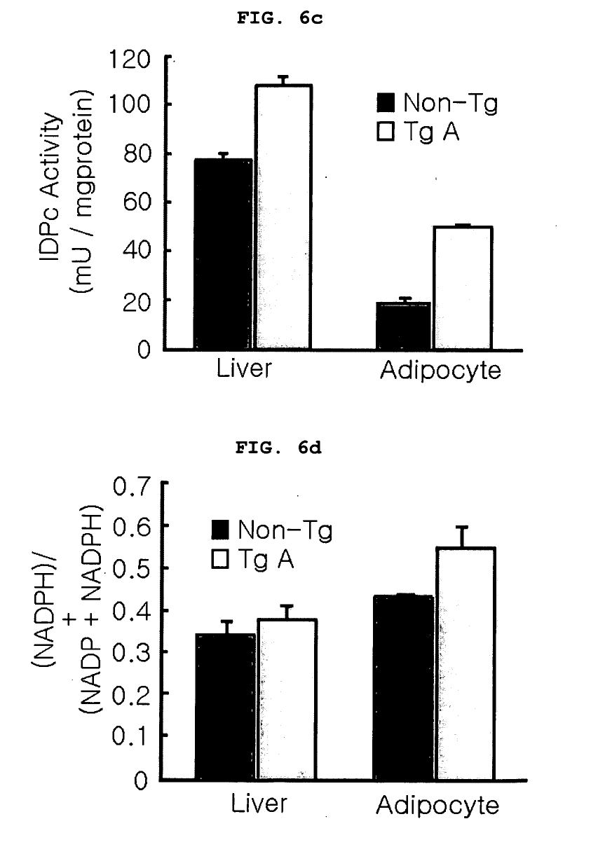

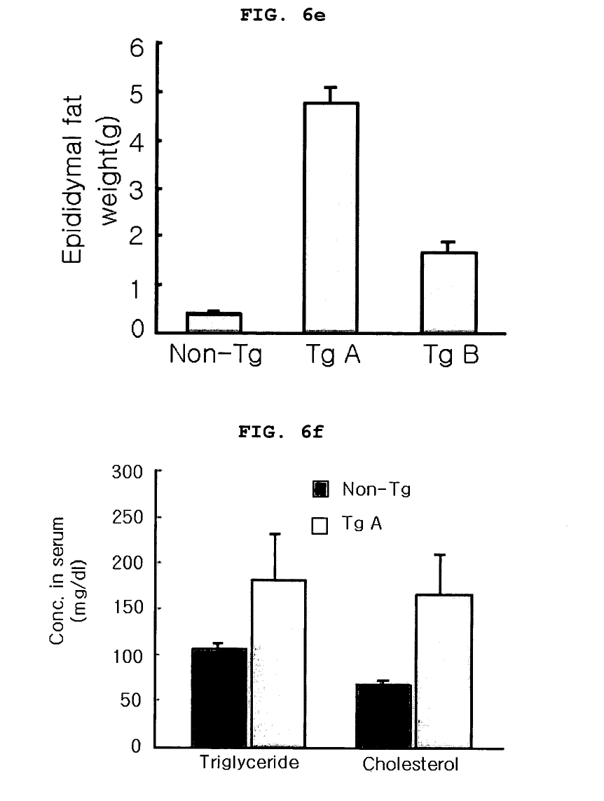

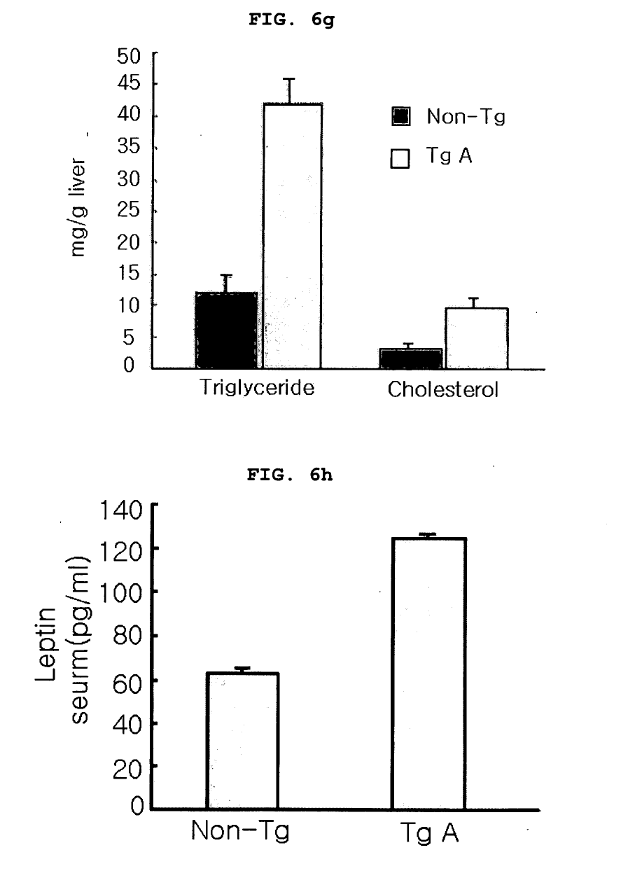

The transgenic mice were found to show IDPc activity 2.7 and 1.4 fold greater in the liver and the epididymal fat pad than in those of control mice (see FIG. 6c), respectively. Accordingly, the ratio of NADPH to total NADP pool ([NADPH]/[NADP⁺]+[NADPH]) increased with increasing the enzymatic activity of IDPc (see FIG. 6d). The weight of the transgenic mice was increased by 35% or larger compared to that of the normal mice (see FIG. 6a) with a more significant increase in the epididymal fat pad of the transgenic mice than in that of control mice (see FIG. 6e). However, no changes were found in the weight of the liver (see FIG. 6b).

In addition, triglyceride and total cholesterol levels in blood of the transgenic mice were measured to be 1.8 and 2.4 fold greater than those of the control mice (see FIG. 6f). Like blood, the liver of the transgenic mice was increased in both triglyceride and cholesterol levels (see FIG. 6g). Leptin, a protein produced mainly from mast cells, was detected to be twice as high in the blood level of the transgenic mice as in that of the control mice (see FIG. 6h).

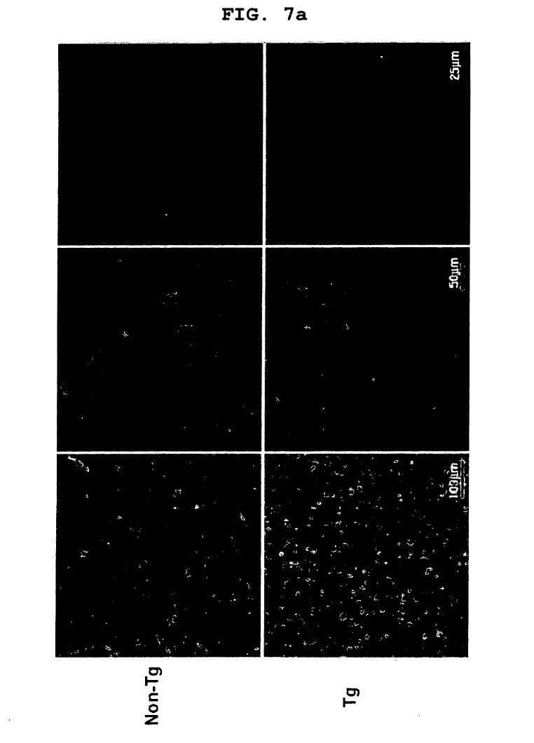

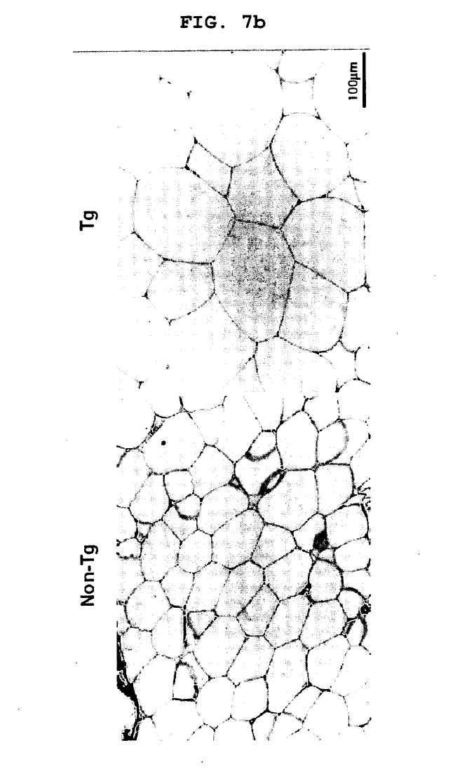

Further, the transgenic mice were observed to have livers in which a greater quantity of fats were deposited compared to those of the control mice. Another significant increase in the transgenic mice over the control mice was found to be the size of adipocytes in the epididymal fat pad (see FIGS. 7a and 7b).

As explained above, the weight gain of the transgenic mice that show more active expression of the IDPc gene is attributed to an increase in the quantity of body fat, and various obesity-indicative genes are more actively expressed in adipose tissues of the transgenic mice than in those of the control mice. For example, there was a significant increase in the level of PPARγ, a transcription factor activating the transcription of genes coding for the enzymes which are responsible for the biosynthesis of lipids. Therefore, an increase in the expression of the IDPc gene primarily results in the production of greater quantities of NADPH which is necessary for the biosynthesis of fatty acids and allows thus abundant lipid derivatives to induce the activation and gene expression of PPARγ, which, in turn, activates the expression of obesity-indicative genes and finally cause obesity in the IDPc-transgenic mice. Meanwhile, an increase in the gene expression and activity of IDPc and in the cellular level of NADPH increases the activity of the enzymes that are involved in the biosynthesis of cholesterol, as well as activating the biosynthesis of lipoproteins through the increasing of lipid levels to produce more quantities of cholesterol composites in which cholesterol is associated with lipoproteins.

In a further aspect, the present invention pertains to a method for screening materials inhibitory of the enzymatic activity and gene expression of isocitrate dehydrogenase and thus effective for the treatment of metabolic diseases such as obesity, hyperlipidemia, and fatty liver.

Based on the enzymatic functions disclosed above, the present invention suggests therapeutics for the treatment of metabolic diseases caused by an increase in fat levels in vivo, such as obesity, hyperlipidemia and fatty liver, by taking advantage of the fact that an increase in the enzymatic activity and gene expression of isocitrate dehydrogenase promotes the biosynthesis of NADPH, in turn, activating PPARγ and raising in vivo levels of fatty acids, squalene and cholesterol.

In this connection, spectrophotometry is very useful. In more detail, first, a spectrophotometer is adjusted to zero absorbance at 340 nm using a mixture of a ten-fold concentrated reaction buffer plus 3rd distilled water. After a crystal cuvette containing the 10× buffer, a test sample and 3rd distilled water is installed in the spectrophotometer, isocitrate dehydrogenase is added to the cuvette and a measurement is made of the change in absorbance at 340 nm with time.

Because an absorbance decreases faster in the cuvette containing a greater concentration of a sample inhibitory against the activity of the enzyme, the analysis of the spectrophotometric data enables the screening of inhibitors of isocitrate dehydrogenase.

Five samples, i.e. nicotinic acid, nicotine amide, bupropion, methyl isocitric acid and oxalomaic acid, were tested for inhibitory activity against isocitrate dehydrogenase. No inhibitory activity was found in the first two samples while bupropion showed a little inhibitory effect. In contrast, methylisocitric acid and oxalomalic acid were measured to have definite inhibitory activity against isocitrate dehydrogenase activity.

In a still further aspect, the present invention pertains to the use of NADPH in promoting the biosynthesis of lipids, cholesterol and squalene and activating PPARγ on the basis of the first finding of the present invention that an artificial increase in the cellular level of NADPH gives great rise to obesity and hyperlipidemia and raises the cellular level of triglyceride.

### EXAMPLES

A better understanding of the present invention may be obtained in light of the following examples which are set forth to illustrate, but are not to be construed to limit the present invention.

### Example 1

### Isolation and Sequencing of IDPc Gene

1-1. Isolation of Mouse IDPc cDNA

A probe for identifying a mouse IDPc cDNA was prepared using the rat IDPc cDNA recently reported (Jennings et al., J. Biol. Chem., 1.69, 21328-23134, 1994). A sense primer listed in Sequence No. 1 was synthesized on the basis of nt 532-550 of the rat IDPc gene and an antisense primer listed in Sequence No. 2 on the basis of nt 1263-1245. Separately, mRNA isolated from rat liver was converted into cDNA by use of reverse transcriptase. Using the primers, a PCR started with 94° C. pre-denaturation for 4 min and carried out with 25 cycles of denaturing at 94° C. for 1 min, annealing at 50° C. for 1 min and extending at 72° C. for 2 min, finally followed by 72° C. extension for an additional 10 min, while 100 ng of the cDNA library was used as a template. As a result, a 0.8 kb DNA sequence was amplified. This PCR product was cloned into pCR II (Invitrogen Co.). The inserts of clones were sequenced to identify a rat IDPc gene.

Molecular cloning of a mouse IDPc cDNA was started with the plaque hybridization of a cDNA library of mouse NIH3T3 cells (Stratagene) with the rat IDPc gene labeled with [α-32P]dCTP- as a probe. All hybridization procedures and washing were conducted at 65° C. F or primary screening, cDNA library phage with 5×10⁴ PFU was mixed with 3×10⁸ cells of E. coli XL1-blue at 37° C. for 15 min. After being well mixed with 7 ml of soft agarose medium (0.7% agarose, 1% tryptone, 0.5% yeast extract, 1% NaCl), the mixture of phage and host was poured in a 150 mm TYM-Ap agarose plate (1% tryptone, 0.5% yeast extract, 1% NaCl, 1.5% agar, 10 mM MgSO₄, ampicillin 50 μg/ml), solidified, and incubated at 37° C. for 12 hours. Following the formation of phage plaques, the plate was stored at 4° C. for 1 hour and then, the phages were transferred onto a nitrocellulose membrane. For use in this plaque hybridization, the nitrocellulose membrane had been soaked in distilled water and 1 M NaCl, in sequence, and dried on a 3 MM filter paper. The phage-coated soft agar plate was covered with the nitrocellulose membrane and then with another one for duplication. These duplicate membranes were immersed in a denaturation buffer (0.5 M NaOH, 0.5 M NaCl) for 5 min to denature the phages from host cell lysis, and phage DNA, and allowed to stand in a neutralization buffer (0.5 M Tris-Cl, pH 8.0, 0.5 M NaCl) for 5 min, finally followed by drying on a 3 MM filter paper.

Baking at 80° C. for 2 hours immobilized the phage DNA onto the membrane which was then washed with 6×SSC (1×SSC; 0.15 M NaCl, 0.015 M sodium citrate, pH 7.0) containing 1% SDS, followed by pre-hybridization for 2 hours at 65° C. Using the [α-³²P] dCTP-labeled rat IDPc gene as a probe, hybridization was conducted in 6×SSC solution containing 5× Denhardt solution (0.1% ficoll, 0.1% polyvinylpyrrolidone, 0.1% BSA), salmon sperm DNA 100 μg/ml, and 0.01% SDS. After completion of the hybridization, the membrane was kept in direct contact with an X-ray film for 12 hours. The resulting autoradiogram enabled the isolation of single plaques.

Six DNA clones extracted from independent phages were cut with EcoR1, and subjected to Southern blotting using the [α-³²P]dCTP-labeled rat IDPc gene as a probe. These 6 phage clones were identified to have fragments with sizes ranging from 1.9 to 2.2 kb. From among them, the largest cDNA fragment was selected to isolate the mouse IDPc cDNA fragment, after which it was sub-cloned into a pGEM7(+) vector (Promega).

1-2: Sequencing of Mouse IDPc cDNA

The mouse IDPc cDNA gene isolated in Example 1-1 was analyzed for base sequence with the aid of Sequenase version 2.0 kit (United States Biochemicals). From the obtained base sequence, the amino acid sequence was determined. The GeneBank database was used to search for similar amino acid sequences and compared for homology.

From the DNA base sequencing analysis data, it was found that the mouse IDPc cDNA has a 1,245 bp ORF listed in Sequence No. 3 with the sequence AATAAA, which is regarded as a poly(A)+ signal, existing in the 3′-UTR. The amino acid sequence deduced from the base sequence of the IDPc gene is described to consist of 414 residues with a molecular weight of 46,575 Da, as shown in Sequence No. 4.

Alignment of the mouse IDPc and other species-derived isocitrate dehydrogenase proteins indicated that the mouse IDPc of the present invention shares a homology of 97.8% with rat IDPc, 68.5% with bovine IDPm, and 64.4% with yeast IDPc.

In the mouse IDPc amino acid, the sequence nt 412-414 is found to be identical to a peroxisome targeting sequence. Peroxisome is known to be involved in the biosynthesis and degradation of fatty acids and cholesterol. These results offer the high probability that IDPc moves to peroxisomes and takes part in the synthesis of fatty acids and cholesterol thereat.

### Example 2

### Construction of Cell lines Transformed with IDPc Genes

2-1: Construction of Recombinant Retroviral Vector for Expressing IDPc Genes

For the expression of the IDPc gene in cells, the IDPc cDNA obtained in Example 1 was subcloned into the retroviral vector pLNCX (Miller, A. D. and Rosman, G. T., Biotechniques, 7, 980-990, 1989) in sense and antisense orientations.

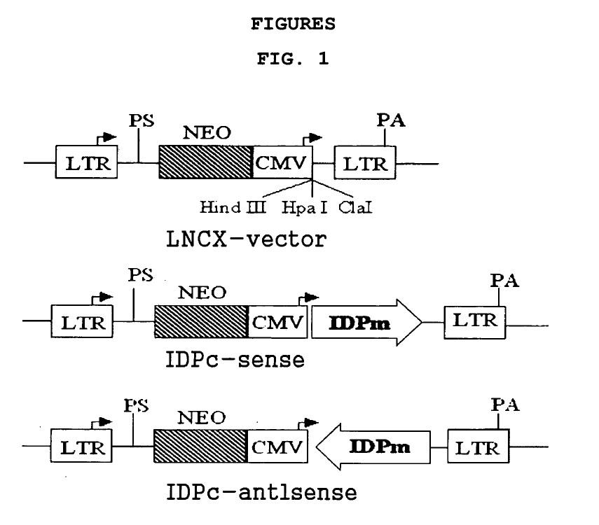

After the IDPc cDNA was cut at its both ends with ClaI, the DNA cut was ligated into the retroviral vector. The recombinant plasmid was introduced into E. coli DH5α and amplified by culturing the microorganism. By use of restriction enzyme digestion, the orientation of the inserts of the clones was identified. In the resulting recombinant vector constructs, the expression of sense or antisense IDPc cDNAs was directed by the cytomegalovirus promoter, as shown in FIG. 1. The recombinant vectors in which the IDPc cDNA was inserted in the sense directions as determined by restriction enzyme digestion, were used to enhance the expression of the gene of interest, while the recombinant vector in which the IDPc cDNA was in the antisense direction were used to restrain the expression of the gene. As a control was used a retroviral vector pLNCX that did not anchor the gene. The recombinant vectors thus obtained were introduced into mouse NIH3T3, a fibroblast cell line. As a marker to identify this transfection, a pLNCX vector containing a GFP (green fluorescence protein) cDNA was also introduced into the cell, simultaneously.

2-2: Construction of Transfectant Cell Lines

The transfection of the recombinant vectors into NIH3T3 cells was achieved by use of retroviral package systems employing BOSC23 cells. In this regard, BOSC23 cells were inoculated at a density of 2×10⁶ cells/ml in DMEM (Dulbecco's Modified Eagle Media) supplemented with 10% FBS and then maintained in DMEM containing 25 μM chloroquin and 10% FBS before use in transfection. The transfection followed the calcium phosphate method (Pear, W. S. et al., Proc. Natl. Acad. Sci. USA, 90, 8392-8396, 1993). After being mixed with 2×HBS (20 mM NaCl, 1.5 mM Na₂HPO₄, 50 mM HEPES, pH 7.1), a solution containing 10 μg of the recombinant vector DNA and 0.25 M CaCl₂ was uniformly added to a plate on which BOSC23 cells were grown, and incubated in a CO₂ incubator. After 10 hours of incubation, the cells were provided with fresh DMEM supplemented with 10% FBS and cultured for 24 hours. Only the medium was centrifuged at 1,200 rpm and the supernatant was filtered through a 45 μm filter. To the filtrate which contained the recombinant retroviral vector only, polybrene (Sigma) was added to a concentration of 4 μg/ml.

In preparation for the transfection of the recombinant retroviral vector, NIH3T3 cells were inoculated at a density of 5×10⁵ cells/ml and cultured in a 10% FBS-supplemented DMEM. When the number of the cells increased by 50%, the medium was removed and the retrovirus particles separated from the packaging cells were added to the NIH3T3. After 5 hours of incubation, the cells were provided with fresh medium and cultured for 2 days.

Counts of the NIH3T3 infected by the recombinant retrovirions were measured, followed by aliquoting the cells at a density of 50 cells per well into 96-well plates in which DMEM added with G-418 (Gibco BRL) 400 μg/ml was contained. While the G-418 medium was changed every other day, the NIH3T3 cells in each well were re-separated and cultured to select first NIH3T3 transformants. For secondary screening, PCR with genomic DNA was performed to verify the integration of IDPc cDNA into the genome.

Among the cells which were finally identified to have an IDPc gene in their genome, the transformants into which the IDPc gene was inserted in the sense direction and the antisense direction, were named FS1 and FAS1, respectively. The cell line FS1, which harbors the IDPc gene in the sense direction in its genome, was deposited with the Korean Collection for Type Culture of Korea Research Institute of Bioscience and Biotechnology (KRIBB) under the deposition No. KCTC 0861BP on Sep. 6, 2000.

### Example 3

### Enzyme Activity of IDPc and IDPm in Transfectant Cells

For the determination of the enzyme activity of IDPc in the transfectant NIH3T3 cells, the cytoplasm was separated from the cells, and concentration of protein was determined using the Bradford assay. First, 3×10⁷ cells/ml were washed twice with 1×PBS and lysed with a sucrose buffer (0.32 M sucrose, 0.01 M Tris-Cl, pH 7.4). The cell lysate was centrifuged at 1,000×g to remove cell debris and then at 15,000×g to pellet mitochondria. To the removed supernatant containing the cytoplasmic fraction, PBS containing 0.1% Triton X-100 was added at 1/10 the total solution volume, followed by quantification by use of the Bradford assay. The enzyme activity of IDPc was determined by measuring the change in the production amount of NADPH in a buffer (50 mM MOPS, pH 7.2, 35.5 mM triethanolamine, pH 7.2, 2 mM NADP⁺, 2 mM MgCl₂, 5 mM isocitrate, and rotenone 1 μg/ml) maintained at 25° C. Using a spectrophotometer, absorbance at 340 nm was measured for 2 min to quantify the amount of NADPH produced by the IDPc contained in the cytoplasmic protein, thereby determining the enzyme activity of IDPc. For the quantification of the enzyme, the amount which could produce 1 μM of NADPH in 1 min was defined as 1 unit.

Compared to the control cells in which only LNCX-vector was introduced, the enzyme activity of IDPc was increased by a factor of about 2 in the transfectant FS1 cells into which the IDPc gene was introduced in the sense direction while being decreased by a factor of about 0.4 in the transfectant FAS1 cells into which the IDPc gene was introduced in the antisense direction.

### Example 4

### Synthesis of Fatty Acid in Transfectant Cell Line According to IDPc Activity

To examine the influence of IDPc on the synthesis of fatty acids, the transfectant cell lines were cultured in 10% FBS-supplemented DMEM containing insulin 5 μg/ml, 0.5 mM 3-isobutyl-1-methylxanthine (IBMX, Sigma), 1 μM dexamethasone (DEX), and penicillin-streptomycin (Gibco BRL), each 50,000 units, for two days to increase the counts of the cells to a density of approximately 3×10⁴ cells/cm². Thereafter, the cells were further cultured for 12 days in DMEM free of IBMX and DEX while the medium was refreshed every other day. Cell culturing was carried out in a CO₂ incubator at 37° C. under a wet, 5% CO₂ atmosphere.

After culturing, the cells were treated with Oil-Red-O, which specifically dyes oil, to observe oil deposits formed in adipocytes. In this connection, the medium was depleted, after which 10 ml of a cacodylate buffer (90 mM cacodylate, pH 7.2, 2% formaldehyde, 2.5% glutaraldehyde, 0.025% CaCl₂, 5% sucrose) was added to the cells, which was then allowed to stand at 4° C. for 1 hour. After removal of the buffer, 5 ml of Oil-Red-O in 40% isopropanol was added to the cells and slowly mixed over 1 hour, followed by washing with 40% isopropanol.

With reference to FIG. 2a, there are observations of the adipocytes dyed with Oil-Red-O. As shown in photographs of FIG. 2, the transfectant FS1 cells in which the gene expression of the IDPc is increased, have produced oils at a greater amount than did the control cells. On the other hand, the transfectant FAS1 cells with decreased expression of the IDPc gene show significantly reduced oil deposits relative to the control cells. Photographs magnified at 200× power further show the difference in adipocyte size among the cell groups. These observations indicate that an increase or a decrease in the gene expression and level of IDPc and in the level of NADPH, a metabolic product of the enzyme, has significant influence on the increase or decrease of cellular fat deposits

### Example 5

### Change in Differentiation of Adipocytes and Oil Deposition within Cells According to Concentration of NAPH

The effect of NADPH, a metabolic product of IDPc, on the deposition of cellular oils was quantitatively measured with Oil-Red-O, a dye specific for lipids. While the control cells NIH3T3 L1 into which only pLNCX vector was introduced were cultured under the same conditions as in Example 4, NADPH was added at amounts of 0 μM, 25 μM and 50 μM to the media to differentiate the cells into adipocytes. After the differentiation, the cells were dyed with the Oil-Red-O solution to visualize the oil deposits formed within cells. Visualized results are given in FIG. 2b. As seen in photographs of FIG. 2b, the cells accumulated greater amounts of oils in the presence of external NADPH than in the absence of external NADPH. In addition, more extensive deposition of oils were observed when external NADPH was supplied at greater amounts. Therefore, NADPH, which can be obtained as a reaction product of not only isocitrate isoenzymes IDPc and IDPm, but also glucose-6-phosphate dehydrogenase, 6-phosphogluconate dehydrogenase and malate dehydrogenase, was identified to have direct positive influence on the differentiation of adipocytes and the concomitant deposition of oils even when it was artificially added.

### Example 6

### Identification of In Vivo Activity of IDPc Using Transgenic Animal Containing IDPc Gene in Its Genome

6-1: Creation of Transgenic Mouse

6-1-1: Preparation of Fused Gene Construct for Microinjection

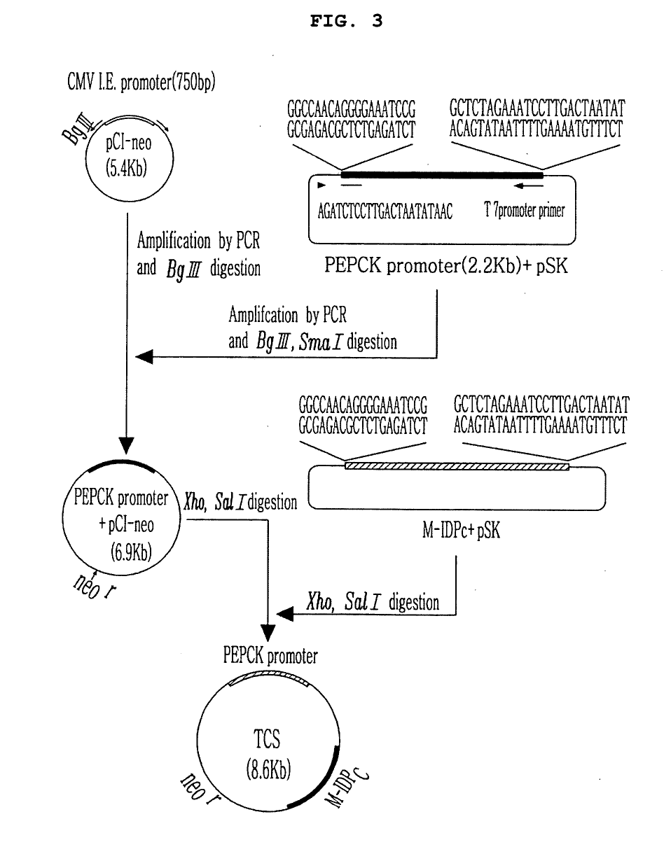

For use in the tissue-specific, permanent expression of the IDPc gene in the liver and adipocytes, a 2.2 kb 5′-upstream sequence containing a promoter was amplified from the cytosolic PEPCK gene of rats with the aid of PfuTurbo DNA polymerase (Stratagene) using a set of primers listed in Sequence Nos. 5 and 6. The PCR product was digested with BglII and SmaI and then with I-Pop-I, and treated with Mung Bean nuclease to produce blunt-ends, one of which was cut with BglII. This DNA digest was inserted into the mammalian expression vector pCI-neo containing a CMV promoter. To this vector, an IDPc cDNA which had been double digested with XhoI and SalI, was ligated. The resulting recombinant vector in which the IDPc gene was expressed under the regulation of the PEPCK 5′-upstream sequence was named pPEPCKIDPc. The recombination procedure was illustrated in FIG. 3.

6-1-2: Preparation of Fused Gene Construct for Microinjection

The mouse IDPc fused gene construct (ca. 10 μg) obtained in Example 6-1-1 was subjected to double digestion with restriction enzymes BglII and NsiI, after which the digestion solution was resolved on 0.7% agarose gel to separate a 4.9 kb DNA fragment containing the gene of interest. The excised gel portion containing the DNA fragment was treated with a mixture of phenol and CIAA (chloroform:isoamyl alcohol=24:1 v/v) in the volume proportion of 1:1. The upper part was centrifuged at 12,000 rpm for 3 min. To the supernatant recovered, an equal volume of ether was added, followed by centrifugation at 10,000 rpm for 5 sec. After the removal of the upper ether part, the lower DNA part was added with two volumes of absolute ethanol to precipitate DNA. The DNA pellet was well dissolved in a microinjection solution (10 mM Tris pH 7.4, 0.1 mM EDTA) at a concentration of 2-10 μg/2.4 ml. The resulting solution was dialyzed against a microinjection solution at 4° C. for 24 hours. For microinjection, the DNA was controlled to have a concentration of 2-4 ng/μl in total and stored at −20° C. until use.

6-1-3: Preparation of Embryo

To FVB/N lineage mice, which produce many harvestable ova and whose embryos suffer little damage upon microinjection, pregnant male's serum gonadotropin (PMSG) and human chorionic gonadotropin (hCG) were peritoneally injected at a dose of 5 IU, each, to induce superovulation. 20 hours after the injection of PMSG and hCG, the mouse oviduct ampulla was blasted to recover cumulus cell mass which was then deprived of cumulus cells by treatment with hyaluronidase (300 μg/ml) for 3 min. Of them, one-cell stage embryos, in which two eukaryons per cell are observed, were selected for use in microinjection.

While monitoring the nuclear membrane under an inverted microscope equipped with a Normarski differential interference contrast (DIC) lens, the fused gene construct was microinjected into the selected embryos with the aid of a micromanipulator. After completion of microinjection, survivors were cultured in M16 medium at 37° C. in a 5% CO₂ atmosphere in a CO₂ incubator. The resulting mouse embryo containing the IDPc fused gene construct was deposited with the Korean Collection for Type Culture of Korea Research Institute of Bioscience and Biotechnology (KRIBB) under the deposition No. KCTC 0874 BP, on Nov. 5, 2000.

6-1-4: Implantation of Mouse Embryo and Identification of Transgenic Mice

By virtue of their large uterine area and high proliferation and suckling capability, FVB/N lineage mice were used as recipients. Vasectomy was performed on male mice in the estrus stage, which were allowed to mate with females. The next morning, the female mice which showed vaginal plugs were selected as final recipients. Using scissors, dissection was performed at the subcutis of the sperm duct of the recipients to the length of 1 cm and subsequently at the muscle layer, followed by implanting the embryo of Example 6-1-3 into the opposite oviduct thus exposed.

Offspring from the recipients were examined by PCR for the insertion of the microinjected mouse IDPc fused gene construct DNA, that is, pPEPCKIDPc, into their genome. To this end, a tail part 2-3 cm long was cut from 2 or 3-week-old mice bred by the recipient, and immersed in 700 μl of a lysis buffer (50 mM Tris-Cl, pH 8.0, 100 mM EDTA, 100 mM NaCl, 1% SDS) for 15-18 hours at 55° C. in the presence of 35 μl of proteinase K (10 mg/ml) with agitation. After RNA hydrolysis with 20 μl of RNase (13 μg/ml), the hydrolyzed solution was added with an equal volume of phenol, followed by centrifugation. On the supernatant, the phenol extraction was repeated two or three more times. To the final supernatant were added two volumes of absolute ethanol to precipitate DNA. Serving as a template, the mouse genomic DNA (1 μg) thus obtained was partially amplified by PCR using a 3′-flaking region of the CMV promoter as a sense primer P1 (Sequence No. 7) and a 5′-flanking region of the IDPc gene as an antisense primer P2 (Sequence No. 8). The PCR started with denaturing at 95° C. for 5 min, followed by 30 cycles of denaturing at 95° C. for 1 min, annealing at 51° C. for 1 min and extending at 72° C. for 1.5 min. The PCR solution was resolved on 1.5% agarose gel to select a mouse presenting a 0.5 kb DNA band identifying it as a transgenic mouse. Offspring from the crossing of selected transgenic male mice with wild-type female FVB/N mice had their tails cut and examined for transgenicity in the same manner to select the transgenic mice, which inherited the recombinant IDPc gene in a germ line. After being identified by PCR as having the recombinant gene, the transgenic mouse offspring were maintained in a heterozygous F₂ line. Like the F₁ heterozygous transgenic mice, these F₂ heterozygous transgenic mice were found to show obesity, hyperlipidemia, and fatty liver.

For managing the transgenic mouse species, 2-week-old mice had their tails cut partially and genomic DNA was prepared from the tail segments and analyzed for the insertion of exogenous gene of interest. Once being identified as transgenic, mice were separated according to sex and marked in their ears.

6-2: Weight Gain of Adipose Tissue and IDPc Activity in Transgenic Animal

6-2-1: Enlargement of Epididymal Fat Pad in Transgenic Animal

Transgenic mice and normal mice, both being 28 weeks old and bred from the same parents, were sacrificed by separation of their spines, after which their exodermis was partially dissected with scissors while being hold by a pincette. After the complete peeling of the exodermis, the endodermis was dissected to expose epididymal fat pads which were then compared in size to those of wild-type FVB/N mice. Afterwards, total adipose tissues were taken from both the transgenic mice and wild-type mice in order to compare the total weights therebetween. Soon after being measured for weight, the separated total adipocytes were fixed in formalin and rapidly cooled. Using a microtome, the frozen adipose tissues were sliced at −20° C. to pieces 10 μm thick, which were dyed with hematoxylin and eosin for visualization under a microscope.

Microscopic observations are given in FIG. 4. 26 weeks after birth, as seen in FIG. 4a, F₁ heterozygotic transgenic mice had grown bigger than control mice. When being frayed, the transgenic mice were observed to have large and many dermal mast cells on their backs, compared to control mice, as shown in FIG. 4b. In addition, the transgenic mice were identified to have significantly larger epididymal fat pads. When being further anatomized, F₁ heterozygotic transgenic mice were measured to have significantly larger epididymal fat pads compared to control mice, as shown in FIG. 4c. After complete removal of the abdominal skin, a significant increase in the weight of white adipose tissue was observed in the transgenic mice relative to control mice, as seen in FIG. 4d. No difference in the size and color of the liver between the transgenic mice and the normal mice was seen with the naked eye.

6-2-2: Change in Expression Level of Obesity-Indicative Gene According to Expression of IDPc Gene in Transgenic Mice

To examine whether the expression of IDPc gene has influence on the expression of obesity-indicative genes, each obesity-indicative gene was quantitatively measured as to its expression level as follows.

In detail, 1 g of the epididymal fat pad taken from each of the IDPc gene-transgenic and the normal mice was added in 9 ml of a lysogenic solution (4M guanidium thiocyanate, 25 mM sodium citrate, pH 7.0, 0.5% sarkosyl, 0.72% β-mercaptoethanol) and homogenized by use of a homogenizer. After being cooled for 2-3 min in ice, the homogenate was added with a mixture of 1 ml of an extraction solution (2M sodium acetate, pH 4.0) and 10 ml of DEPC-water saturated phenol, 2 ml of chloroform-isoamyl alcohol (24:1) and let to stand in ice for 15 min. After the solution was centrifuged at 3,000×g at 4° C. for 15 min, the supernatant was extracted again with phenol/chloroform. The extract was added with an equal volume of isopropanol, followed by centrifugation for 15 min to give an RNA pellet. After being dissolved in an aqueous 36% formaldehyde solution, the RNA was resolved on 1% agarose gel containing formaldehyde at an amount of 6.7% under an electric field. Following the electrophoresis, separated RNA bands were transferred onto a nylon membrane in 20×SSC solution, dried and fixed by UV cross-linking. The nylon membrane was washed for 5 min in 6×SSC solution and then subjected to pre-hybridization at 42° C. for 2 hours in an appropriate amount of a hybridization solution (50% formamide, 6×SSC, 5× Denhardt's solution, 1.2% SDS, 10 μg/ml salmon sperm DNA). To be used as probes for Northern blotting, various obesity-indicative cDNAs (dP2, adipsin, LPL (lipo protein lipase), leptin, tumor necrosis factor α[TNFα], and PPARγ) were labeled with [α-³²P]dCTP. Using these radiolabeled probes, hybridization was conducted for 12 hours. After completion of the hybridization, the nylon membrane was washed at 65° C. with 6×SSC solution containing 0.1% SDS for 30 min and then with 2×SSC containing 0.1% SDS for 20 min and additionally washed in the same manner as above at least one more. Finally, the nylon membrane was washed twice with 0.2×SSC solution at room temperature. The autoradiogram obtained by exposing the membrane to X-ray film at −70° C. allowed the identification of mRNAs transcribed from obesity-indicative genes in the epididymal fat pad.

With reference to FIG. 5, the hybridization results are shown in autoradiographs. As seen in the autoradiographs, the expression of the recombinant IDPc gene introduced to the transgenic mice was found, along with the expression of the endogenous IDPc gene, demonstrating that the total IDPc activity was increased. Additionally, an increase was found in the expression of obesity-indicative genes, such as genes coding for aP2, adipsin, LPL, leptin, TNF-α, and peroxisome proliferator-activated receptor γ (PPARγ), which are all known to show increased expression with the advance in the differentiation of mast cells. That is, the increased in IDPc activity due to an increase in the gene expression of IDPc was shown to stimulate the expression of all of the obesity-indicative genes. As demonstrated in FIG. 2b, these results indicate that NADPH is involved in the expression of the obesity-indicative genes.

In light of the recent reports describing the positive feedback mechanism of PPARγ in which the activation of PPARγ further stimulates the expression of its gene (Kim, J. B. et al., Proc. Natl. Acad. Sci. USA, 95, 4333-4337, 1998), along with the reports concerning the function of polyunsaturated fatty acids and lipid derivatives as ligands to activate PPARγ, the above results can be interpreted to mean that an increased cellular level of NADPH attributed to an increase in cellular IDPc activity stimulates the activity of fatty acid syntheses which indispensably require NADPH for their enzymatic reactions, thus raising the cellular level of fatty acids. Additionally, in view of the reports showing that increased cellular levels of fatty acid derivatives resulting from a sufficient supply of NADPH leads to the activation of PPARγ that serves as a master transcription factor to promote not only the expression of genes responsible for the synthesis of fatty acids necessary for the differentiation of mast cells, but also genes encoding proteins involved in the biosynthesis of cholesterol, the increase in the expression of obesity-indicative genes such as gene coding for ap2, adipsin, LPL, leptin, and TNFα is believed to be due to the increased expression of the PPARγ gene. Therefore, there can be obtained a conclusion that the increase in IDPc activity attributed to the active expression of the IDPc gene and a concomitant increase in NADPH level is directed to a sharp increase in the gene expression of PPARγ, which is indispensable for both the differentiation of adipocytes and the biosynthesis of lipids.

6-2-3: Weight Gain and Increase in Intracellular Level of Lipid and Cholesterol According to IDPc Expression in Transgenic Animal and Biochemical Examination Therefor

26 weeks after the birth, obesity transgenic mice F₁ and normal mice were compared for intracellular lipid deposition.

1. Measurement of Body Weight

A container suitable for receiving a mouse was placed on a scale which was then subjected to null adjustment, after which a mouse was carefully put in the container. Because the numeral read on the scale was changed whenever the mouse moved, the value detected when the mouse did not move was set forth as the body weight for the mouse.

2. Determination of Enzyme Activity of IDPc

The livers and adipose tissues taken from the transgenic mice and normal mice were homogenized in a buffer (0.32 M sucrose, 0.01 M Tris-Cl pH 7.4) and centrifuged at 3,000×g for 15 min. Then, the supernatant was recentrifuged at 10,000×g for 15 min. A pure cytosolic fraction was obtained as the supernatant. The enzyme activity of IDPc was determined by measuring the change in the production amount of NADPH in a buffer (50 mM MOPS, pH 7.2, 35.5 mM triethanolamine, pH 7.2, 2 mM NADP⁺, 2 mM MgCl₂, 5 mM isocitrate, and rotenone 1 μg/ml) maintained at 25° C. Using a spectrophotometer, absorbance at 340 nm was measured for 2 min to quantify the amount of NADPH produced by the IDPc contained in the cytoplasmic protein, thereby determining the enzyme activity of IDPc. For the quantification of the enzyme, the amount which produced 1 μM of NADPH for 1 min was defined as 1 unit.

3. Measurement of [NADPH]/[NADPH+NADP⁺]

Quantification of NADPH was based on the principle that NADPH is reacted with MTT (3-[4,5-dimethylthiazol-2-yl]-2,5-diphenyltetrazolium with a concomitant color change. Two cytosolic extracts containing 100 μg of proteins were prepared: one was pre-treated by reaction at 60° C. for 30 min and cooling to 0° C., so as to degrade all NADP⁺ to measure the amount of the preexisting NADPH [NADPH] (sample 1); and the other was stored at 0° C. without pretreatment and used to measure the total NADP pool [NADPH+NADP⁺] (sample 2). Each of the two samples were added to a reaction solution (0.1 M Tris-HCl buffer, pH 8.0, 5 mM EDTA, 2 mM phenazine ethosulfate, 0.5 mM MTT) which was then added with 1.3 units of glucose-6-phosphate dehydrogenase and incubated at 37° C. for 5 min to convert NADPH from all of the NADP⁺existing in the reaction solution. In this regard, the enzyme substrate glucose-6-phosphate was added at an amount of 1 mM to each sample (samples 1 and 2), but not to a control. After completion of the reaction, a change in absorbance at 570 nm resulting from the reaction with MTT was measured to quantify NADPH. Because the absorbance changes detected in samples 1 and 2 were attributed to [NADPH] and [NADPH+NADP⁺], respectively, the ratio of NADPH to total NADP pool ([NADPH]/[NADP⁺]+[NADPH]) could be determined.

4. Quantification of Blood Triglyceride Level and Total Cholesterol

For the measurement of the blood triglyceride levels and total cholesterol, assay kits manufactured by Asan Pharmaceutics. Co. Ltd. were used. Blood samples taken from the transgenic mice and normal mice were treated with an anti-coagulant and centrifuged to obtain sera. 10 μl of each of the sera was mixed with 1.5 ml of a triglyceride-assay kit [a solution of lipoproteinase 10800 U, glycerol kinase 5.4 U, peroxidase 135,000 U, and L-α-glycerophosphate oxidase 160 U in 72 ml of N,N-bis(2-hydroxyethyl)-2-aminomethanesulfonic acid buffer] or 1.5 ml of a cholesterol enzyme-assay kit (a mixture of an enzyme solution (cholesterol esterase 20.5 KU/l, cholesterol oxidase 10.7 KU/l, and sodium hydroxide 1.81 g/l) and a buffer (potassium monophosphate 13.6 g/l, phenol 1.88 g/l) in the proportions of 1:1) and incubated at 37° C. for 5 min for reaction. In a microplate reader, a measurement was made of absorbance at 500 nm for cholesterol and at 540 nm for triglyceride, so as to quantify blood levels of triglyceride and total cholesterol. In connection to the quantification of triglyceride and cholesterol, there was utilized a standard curve which was obtained by applying the above procedure to various known concentrations of a standard solution.

5. Quantification of Blood Triglyceride Level and Total Cholesterol

A predetermined amount of the liver was taken from both the transgenic mice and normal mice and homogenized in 1 ml of CIAA (chloroform:isoamyl alcohol=2:1 v/v). 100 μl of the homogenate was dissolved in 200 μl of pure ethyl alcohol and mixed with 500 μl of each of the assay kits for triglyceride and cholesterol, manufactured by Asan Pharmaceutics, Co. Ltd., containing 0.5% Triton X-100 and 3 mM sodium cholate, followed by incubation at 37° C. for 10 min. After being added with 800 μl of water, each sample was measured for liver triglyceride and total cholesterol levels with the aid of a microplate reader in the same manner as in above. Likewise, the above procedure was applied to various known concentrations of a standard solution to acquire a standard curve which was used to quantify triglyceride and cholesterol levels in the liver.

6. Quantification of Blood Leptin Level

After being coagulated overnight at 2-8° C., blood taken from the transgenic mice and normal mice was centrifuged at 53,000×g for 20 min to separate sera. The sera was frozen at −20° C. until use for concentration measurement of leptin. The blood level of leptin was determined by using an ELISA kit (mouse leptin [OB] calorimetric kit, R&D Systems) according to the manufacturer's protocol. To begin with, 50 μl of the serum was mixed with an equal volume of 2.5 N acetic acid/10 M urea and the mixture was allowed to stand at room temperature for 10 min. For neutralization, 50 μl of 2.7 N NaOH/1 M HEPES was added to the mixture. Before concentration measurement, the resulting sample mixture was diluted 1/20 with a calibrator diluent (RD5-3). 50 μl of an assay diluent (RD1W) was added to each well of 96-well plates, followed by the addition of 50 μl of the serum sample or 50 μl of a reference material. The resulting solution in each well was completely mixed for 1 min and incubated at room temperature for 2 hours to perform the reaction. After complete removal of liquid, each well was washed 4-5 times with a washing buffer. The residue in each well was reacted with 100 μl of a mouse leptin conjugate at room temperature for 2 hours and washed 4-5 times again. Within 30 min, blood leptin levels were quantified by measuring the absorbance at 450 nm in a microplate reader.

Measurements obtained in Example 6-2-3 were analyzed and graphed in FIG. 6. After raising of 26 weeks, as seen in FIG. 6a, the transgenic mice (Tg) were measured to have weights 23-35% higher than the normal mice (Non-Tg). In addition, the transgenic mice were found to show 1.4- and 2.7-fold greater activity of IDPc in the liver and adipose tissues, respectively, than the normal mice, as shown in FIG. 6c. FIG. 6d compares concentration ratios of NADPH to total NADP pool ([NADPH]/[NADP⁺]+[NADPH]) in the liver and the adipose tissue between the transgenic mice and the normal mice. The concentration ratios in the liver and adipose tissue of the transgenic mice was increased by factors of 1.2 and 1.3, respectively, relative to those of the normal mice. A great increase was detected in the weight of epididymal fat pad. The transgenic mice were measured to have epididymal fat pad about 13.6-fold heavier than that of the normal mice as shown in FIG. 6e. However, no significant difference was found in the weight of the liver of the normal mice and transgenic mice, as illustrated in FIG. 6b. As for triglyceride and total cholesterol, the blood levels were higher in the transgenic mice by factors of 1.8 and 2.4, respectively, compared to those in the normal mice, as shown in FIG. 6f. Similarly, triglyceride and total cholesterol levels in the liver of the transgenic mice were 1.8- and 2.4-fold higher respectively, compared to normal mice, as shown in FIG. 6g. FIG. 6h show blood levels of leptin, a protein produced mainly from mast cells are high by a factor of about 2 in the transgenic mice relative to normal mice.

Furthermore, the liver and adipose tissues taken from obesity gene-transgenic mice F₁ and normal mice were observed under a microscope. For observation convenience, the tissues were sliced into sections. The liver of the transgenic mice was identified to be a fatty liver which had accumulated more fat than that of the normal mice, as seen in FIG. 7a. Adipocytes of the transgenic mice were also observed to be five-fold larger in size compared to those of normal mice (FIG. 7b).

As described above, the weight gain of the transgenic mice in which IDPc gene is actively expressed results from body fat accumulation. Additionally, cellular levels of various obesity-indicative proteins, including PPARγ, which is a transcriptional factor promoting the expression of genes encoding enzymes involved in the metabolism of lipids and cholesterol, are significantly elevated in the adipose tissue of the transgenic mice, compared to normal mice. Therefore, an increase in the expression of the IDPc gene primarily results in the production of greater quantities of NADPH, which is necessary for the biosynthesis of fatty acids, and allows the resulting abundant lipid derivatives to induce the activation and gene expression of PPARγ, which, in turn, activates the expression of obesity-indicative genes and finally causes obesity in the IDPc-transgenic mice.

### Example 7

### Screening of Inhibitors against Isocitrate Dehydrogenase

In order to select materials capable of regulating the activity of isocitrate dehydrogenase, the following procedure was performed.

An assay buffer (50 mM MOPS, pH 7.2, 35.5 mM triethanolamine, pH 7.2, 2 mM NADP⁺, 2 mM MgCl₂, 5 mM isocitrate, rotenone 1 mg/ml) and test samples were all prepared at 10× concentration. Enzymatic reactions necessary for the selection were conducted in a final volume of 1 ml at 25° C. in crystal cuvette. For use, the concentrated test samples were diluted with 3rd distilled water. The assay buffer, enzyme inhibitors, and isocitrate dehydrogenase, which all at 10× concentration, were maintained at low temperature, i.e., in ice.

A spectrophotometer was first subjected to null adjustment at 340 nm using the 10× assay buffer and 3rd distilled water. 100 μl of the sample was added, along with 100 μl of the 10× assay buffer, in a crystal cuvette, and mixed with 600 μl of 3rd distilled water. After the completion of the null adjustment, the sample cuvette was installed in the spectrophotometer, followed by adding 200 μl of isocitrate dehydrogenase and mixing the solution with the aid of a pipette. Changes in absorbance at 340 nm were monitored with time. In principle, an absorbance decreases faster in the cuvette containing a greater concentration of a sample inhibitory against the activity of the enzyme. Based on this principle, the quantification of the spectrophotometric data enabled the screening of inhibitors of isocitrate dehydrogenase.

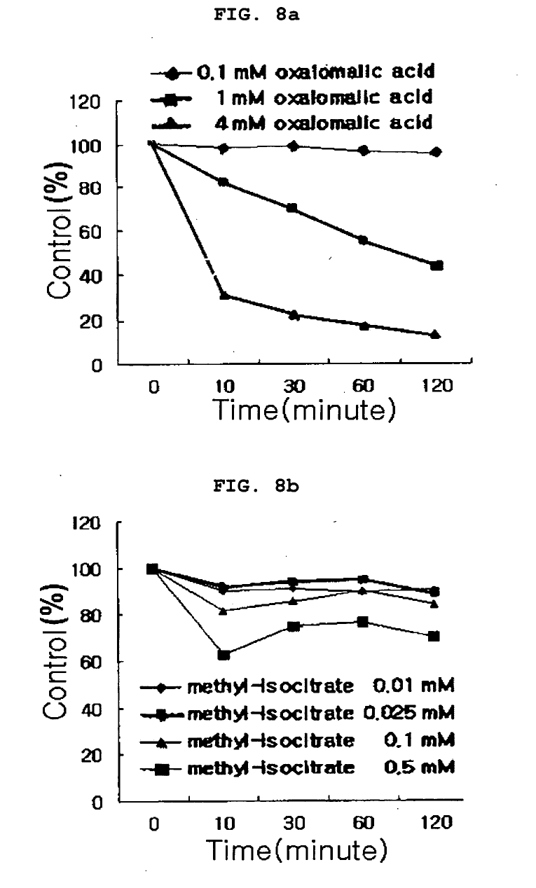

An examination was made of the inhibitory activity against isocitrate dehydrogenase of five samples, i.e. nicotinic acid, nicotine amide, bupropion, methyl isocitric acid and oxalomaic acid. The first two samples were found to show no inhibitory activity while only a little inhibitory activity was detected with bupropion. In contrast, oxalomalic acid (FIG. 8a) and methylisocitric acid (FIG. 8b) were measured to have strong inhibitory activity against isocitrate dehydrogenase.

### Example 8

### Effect of Isocitrate Dehydrogenase Inhibitor on Obesity Prevention

8-1: Prevention of Oil Deposition in 3T3-L1 by Isocitrate Dehydrogenase Inhibitor

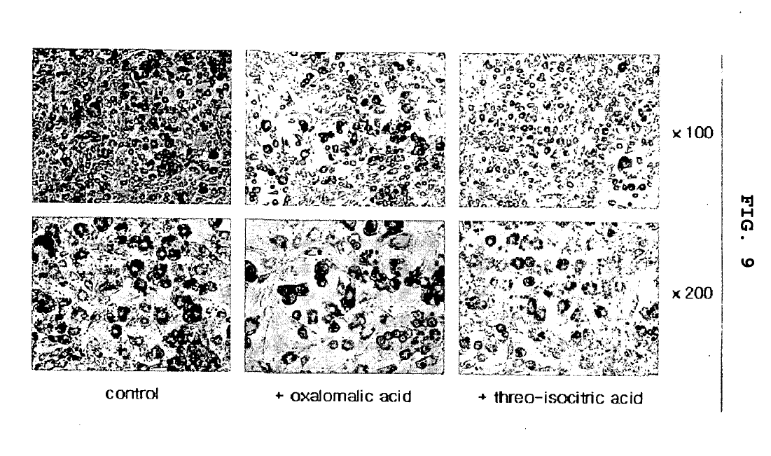

After being treated with the isocitrate dehydrogenase inhibitors methyl isocitric acid and oxalomalic acid, 3T3-L1 cells, which remain undifferentiated, were examined for their differentiation to adipocytes. Effects of the isocitrate dehydrogenase inhibitors on fatty acid synthesis could be identified through the visualization using Oil-Red-O, a oil-specific dye. The results are given in FIG. 9 which shows oil deposits dyed with Oil-Red-O in the cells treated with no isocitrate inhibitors (left), oxalomalic acid (middle), and threo-isocitric acid (right) in photographs with 100 magnification power (upper panel) and 200 magnification power (lower panel). As seen in these optical photographs, IDPc inhibitors play an important role in the lipid metabolism in vivo, reducing cellular oil deposits.

8-2: Restrictive Effect of Isocitrate Dehydrogenase Inhibitor on Obesity in Rats

26 weeks after birth, rats were compared for cellular lipid deposition when they were treated with no isocitrate dehydrogenase inhibitors and methyl isocitric acid.

1. Measurement of Body Weight

A container suitable for receiving a rat was placed on a scale which was then subjected to null adjustment, after which a rat was carefully put in the container. Because the numerals read on the scale changed whenever the rat moved, the value detected when the rat did not move was set forth as the body weight for the rat.

2. Determination of Enzyme Activity of IDPc

The livers and adipose tissues taken from inhibitor-administered rats and non-administered rats were homogenized in a buffer (0.32 M sucrose, 0.01 M Tris-Cl pH 7.4) and centrifuged at 3,000×g for 10 min. Again, the supernatant was centrifuged at 10,000×g for 15 min. A pure cytosolic fraction was obtained as the supernatant. The enzyme activity of IDPc was determined by measuring the change in the production amount of NADPH in a buffer (50 mM MOPS, pH 7.2, 35.5 mM triethanolamine, pH 7.2, 2 mM NADP⁺, 2 mM MgCl₂, 5 mM isocitrate, and rotenone 1 μg/ml) maintained at 25° C. Using a spectrophotometer, absorbance at 340 nm was measured for 2 min to quantify the amount of NADPH produced by the IDPc contained in the cytoplasmic protein, thereby determining the enzyme activity of IDPc. For the quantification of the enzyme, the amount which produced 1 μM of NADPH in 1 min was defined as 1 unit. Protein quantification was performed according to the Bradford assay.

3. Quantification of Blood Level of Triglyceride and Total Cholesterol

In order to measure the blood triglyceride levels and total cholesterol, assay kits manufactured by Asan Pharmaceutics. Co. Ltd. were used. After being treated with an anti-coagulant, blood taken from the inhibitor-administered rats and non-administered rats was centrifuged to obtain sera. 10 μl of each of the sera was mixed with 1.5 ml of a triglyceride-assay kit [a solution of lipoproteinase 10800 U, glycerol kinase 5.4 U, peroxidase 135000 U, and L-α-glycerophosphate oxidase 160 U in 72 ml of N,N-bis(2-hydroxyethyl)-2-aminomethanesulfonic acid buffer] or 1.5 ml of a cholesterol enzyme-assay kit (a mixture of an enzyme solution (cholesterol esterase 20.5 KU/l, cholesterol oxidase 10.7 KU/l, and sodium hydroxide 1.81 g/l) and a buffer (potassium monophosphate 13.6 g/l, phenol 1.88 g/l) in the proportions of 1:1) and incubated at 37° C. for 5 min for reaction. In a microplate reader, a measurement was done by reading absorbance at 500 nm for cholesterol and at 540 nm for triglyceride, so as to quantify blood levels of triglyceride and total cholesterol. In connection to the quantification of triglyceride and cholesterol, there was utilized a standard curve which was obtained by applying the above procedure to various known concentrations of a standard solution.

4. Quantification of Liver Level of Triglyceride and Total Cholesterol

A predetermined amount of the liver was taken from both the inhibitor-administered mice and non-administered mice and homogenized in 1 ml of CIAA (chloroform:isoamyl alcohol=2:1 v/v). 100 μl of the homogenate was dissolved in 200 μl of pure ethyl alcohol and mixed with 500 μl of each of the assay kits for triglyceride and cholesterol, manufactured by Asan Pharmaceutics, Co. Ltd., containing 0.5% Triton X-100 and 3 mM sodium cholate, followed by incubation at 37° C. for 10 min. After being added with 800 μl of water, each sample was measured for liver triglyceride and total cholesterol levels with the aid of a microplate reader in the same manner as in above. Likewise, the above procedure was applied to various known concentrations of a standard solution to acquire a standard curve which was used to quantify triglyceride and cholesterol levels in the liver.

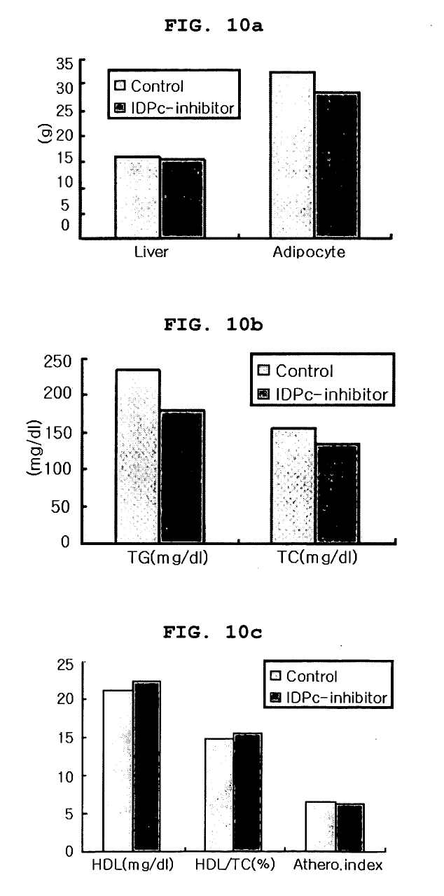

Measurements obtained in Example 8-2 were analyzed and graphed in FIG. 10. No significant difference was found in body weight between the inhibitor-administered and non-administered rats, both being 10-weeks old. As seen in FIG. 10a, the inhibitor-administered rats were measured to be lower in the weight of epididymal fat pad by about 12%, compared to the non-administered rats, with no difference in the weight of the liver of the normal and inhibitor administered rats. As for triglyceride and total cholesterol, their blood levels were lower in the inhibitor-administered rats by 20% and 11%, respectively, compared to those in the non-administered rats, as shown in FIG. 10b. Also, the arteriosclerosis index was 10 lower. On the other hand, high-density lipoproteins (HDL), which transport cholesterol from tissues to the liver through cholesterol counter-transport pathways and thus act to aid the degradation and discharge of cholesterol, was detected at a 11% higher level in the inhibitor-administered rats, compared to the non-administered rats, as shown in FIG. 10c.

As described above, IDPc-inhibitor administered rats are reduced in the level of epididymal fat pads as well as in the blood level of triglyceride and total cholesterol. Additionally, a decrease in arteriosclerosis index is found in the IDPc-inhibitor administered rats. Taken together, these results indicate that IDPc-inhibitors have negative influence on the obesity of animals and reduce the possibility of causing arteriosclerosis. Furthermore, IDPc inhibitors are identified to restrain hypercholesterolemia as they increased the cellular level of HDL, which are involved in the degradation and discharge of cholesterol.

In the present invention, it is disclosed that the weight gain of the transgenic mice in which IDPc gene is actively expressed results from body fats accumulation and that cellular levels of various obesity-indicative proteins, including PPARγ, which is a transcriptional factor promoting the expression of genes encoding enzymes involved in the metabolism of lipids and cholesterol, are significantly elevated in the adipose tissue of the transgenic mice, compared to in that of the normal mice. Therefore, an increase in the expression of the IDPc gene primarily results in the production of greater quantities of NADPH, which is necessary for the biosynthesis of fatty acids, and allows the resulting abundant lipid derivatives to induce the activation and gene expression of PPARγ, which, in turn, activates the expression of obesity-indicative genes and finally causes obesity in the IDPc-transgenic mice.

Based on these research results, IDPc inhibitors were demonstrated to be useful in treating metabolic diseases. That is, when rats are administered with isocitrate dehydrogenase inhibitors, they are restricted from being obese in addition to showing small epididymal fat pads, low levels of triglyceride and total cholesterol, and low arteriosclerosis indexes. Therefore, isocitrate dehydrogenase inhibitors can suppress obesity-induced arteriosclerosis. Also, the increase of HDL level attributed to IDPc inhibitors demonstrates that the decrease in the level of total cholesterol upon the administration of IDPc inhibitors is thanks to the increase of HDL level.

### INDUSTRIAL APPLICABILITY

In the present invention, as described before, the expression of the IDPc gene and the concomitant increase in IDPc level is identified to bring about an increase in the cellular level of NADPH, which, in turn, causes the lipid deposition in adipocytes, leading to obesity and fatty liver. Concurrently, it is also found that blood triglyceride levels and total cholesterol are increased as a result of the expression of the IDPc gene. On the other hand, a decrease in the cellular level of NADPH, resulting from the suppression of the gene expression of IDPc and concurrent intracellular decrease of IDPc levels, has the effect of inhibiting the lipid deposition in adipocytes. Therefore, IDPc gene, IDPc and NADPH can be used for the synthesis of lipids, including fatty acids, squalene, DHA, etc., and for the activation of PPARγ. In addition, because the IDPc-gene transgenic mice of the present invention clearly exhibit symptoms of obesity, hyperlipidemia and fatty liver, NADPH-producing isocitrate dehydrogenase, including IDPc, and their genes can be directly used to identify materials suppressive of obesity and fatty liver as well as materials inhibitory of the biosynthesis of triglyceride and cholesterol. Further, by taking advantage of the suppressive or inhibitory effects of isocitrate dehydrogenase inhibitors on obesity and glyceride and cholesterol biosynthesis, pharmaceutically effective materials for the prophylaxis and treatment of obesity, hyperlipidemia and fatty liver can be developed.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

What is claimed is:

1. An isocitrate dehydrogenase for catalyzing the production of NADPH, useful in the biosynthesis of fatty acids and cholesterol and the deposition of fats.

2. The isocitrate dehydrogenase as set forth in claim 1, wherein the isocitrate dehydrogenase has a mouse-derived amino acid sequence represented by Sequence No. 4.

3. A gene, having a base sequence represented by Sequence No. 3, which encodes the isocitrate dehydrogenase of claim 1.

4. A fused gene construct, comprising the gene of claim 3 inserted in the sense direction therein.

5. A fused gene construct, comprising the gene of claim 3 inserted in the antisense direction therein.

6. A cell strain (Deposition No. KCTC 0861 BP), transformed with the fused gene construct of claim 4.

7. A fused gene construct, based on the gene map of FIG. 3, having the gene of claim 3, wherein the gene is inserted in the sense direction downstream of a rat cytosolic phosphoenolpyruvate carboxykinase gene promoter.

8. An embryo (Deposition No. KCTC 0874 BP), containing the fused gene construct of claim 7.

9. A transgenic animal, harboring the fused gene construct of claim 7 in its genome.

10. The transgenic animal as set forth in claim 9, wherein said animal is a mouse.

11. An agent for promoting the biosynthesis of NADPH, comprising the isocitrate dehydrogenase of claim 1 or the gene of claim 3 as an effective ingredient.

12. An agent for activating the activity of peroxisome proliferator-activated receptor γ (PPARγ), comprising the isocitrate dehydrogenase of claim 1, the gene of claim 3, or NADPH, product of these genes as an effective ingredient.

13. An agent for promoting the biosynthesis of lipids, squalene or cholesterol, comprising the isocitrate dehydrogenase of claim 1 or gene of claim 3.

14. An agent for the prophylaxis and treatment of obesity, hyperlipidemia, or fatty liver, comprising the gene of claim 3 as a therapeutically active ingredient.

15. Use of NADPH in promoting the biosynthesis of triglycerides, cholesterol, and squalene.

16. A method for promoting the biosynthesis of triglycerides, cholesterol and squalene, in which NADPH, product of isocitrate dehydrogenase of claim 1 is added in vivo.

17. A method for screening an inhibitor against the deposition of fats and the production of triglycerides and cholesterol, in which advantage is taken of the ability of the inhibitor to react with isocitrate dehydrogenase to decrease the enzymatic activity of the isocitrate dehydrogenase, thereby lowering the cellular level of NADPH.

18. A method for screening an inhibitor against the deposition of fats and the production of triglycerides and cholesterol, in which advantage is taken of the ability of the inhibitor to associate with a gene coding for isocitrate dehydrogenase to suppress the expression of the gene, thereby lowering the cellular level of NADPH.

19. A method for screening a material regulatory of the activity of isocitrate dehydrogenase in vitro, in which advantage is taken of the ability of the material to suppress the production of NADPH in the enzymatic reaction system comprising isocitrate dehydrogenase, isocitrate as an enzyme substrate, and NADP⁺ as an coenzyme.

20. A method for screening a material regulatory of the activity of isocitrate dehydrogenase in vivo, in which advantage is taken of the ability of the material to suppress the production of NADPH in a culture medium containing an animal cell line transformed with a gene coding for the isocitrate dehydrogenase.

21. A method for screening a material regulatory of the activity of isocitrate dehydrogenase in vivo, in which advantage is taken of the ability of the material to suppress the production of NADPH in an animal harboring an isocitrate dehydrogenase gene in its genome.

22. A method for treating metabolic diseases, in which a material capable of reacting with isocitrate dehydrogenase to decrease the enzymatic activity is used as a therapeutic and the metabolic disease are obesity, hyperlipidemia and fatty liver.

23. A method for treating metabolic diseases, in which a material capable of associating with a gene coding for isocitrate dehydrogenase to inhibit the activity of the enzyme is used as a therapeutic and the metabolic diseases are obesity, hyperlipidemia and fatty liver.
